(19) United States
(12) Patent Application Publication
(10) Pub. No.: US 2003/0113293 A1
(43) Pub. Date: Jun. 19, 2003

(54) Compositions and methods for delivery of an agent using attenuated Salmonella containing phage

(75) Inventors: David G. Bermudes, Wallingford, CT (US); Ivan C. King, North Haven, CT (US); Caroline A. Clairmont, Cheshire, CT (US)

Correspondence Address: PENNIE AND EDMONDS, 1155 AVENUE OF THE AMERICAS, NEW YORK, NY, 100362711

(73) Assignee: Vion Pharmaceuticals, Inc.

(21) Appl. No.: 10/076,117

(22) Filed: Feb. 13, 2002

Related U.S. Application Data
(63) Continuation of application No. 09/645,418, filed on Aug. 24, 2000, now abandoned.
(60) Provisional application No. 60/150,928, filed on Aug. 26, 1999.

(51) Int. Cl. A61K 48/00; C12N 1/21

(52) U.S. Cl. 424/93.2; 435/252.3

(57) ABSTRACT

The present application generally discloses delivery of an agent which can be therapeutic or prophylactic and, more particularly, the preparation and use of attenuated bacteria, such as Salmonella, containing a bacteriophage in which the genome of the bacteriophage has been modified to encode for a gene product of interest, e.g., an antigen or an anti-tumor protein. The bacteria functions as a vector for delivering the bacteriophage encoded gene product of interest to an appropriate site of action, e.g., the site of a solid tumor.

The present application is a continuation application of U.S. application Ser. No. 09/645,418, filed Aug. 24, 2000, which claims priority to U.S. Provisional Application No. 60/150,928, filed Aug. 26, 1999, the disclosures of which are incorporated by reference herein in their entirety.

### 1. FIELD OF THE INVENTION

The present invention is generally concerned with delivery of an agent which can be therapeutic or prophylactic and, more particularly, with the preparation and use of attenuated bacteria containing a bacteriophage in which the genome of the bacteriophage has been modified to encode for a gene product of interest, e.g., an antigen or an anti-tumor protein. The bacteria functions as a vector for delivering the bacteriophage encoded gene product of interest to an appropriate site of action, e.g., the site of a solid tumor.

### 2. BACKGROUND OF THE INVENTION

A major problem in the chemotherapy of solid tumor cancers is delivery of therapeutic agents, such as drugs, in sufficient concentrations to eradicate tumor cells while at the same time minimizing damage to normal cells. Thus, studies in many laboratories are directed toward the design of biological delivery systems, such as antibodies, cytokines, and viruses for targeted delivery of drugs, pro-drug converting enzymes, and/or genes into tumor cells. Houghton and Colt, 1993, New Perspectives in Cancer Diagnosis and Management 1: 65-70; de Palazzo, et al., 1992a, Cell. Immunol. 142:338-347; de Palazzo et al., 1992b, Cancer Res. 52: 5713-5719; Weiner, et al., 1993a, J. Immunotherapy 13:110-116; Weiner et al., 1993b, J. Immunol. 151:2877-2886; Adams et al., 1993, Cancer Res. 53:4026-4034; Fanger et al., 1990, FASEB J. 4:2846-2849; Fanger et al., 1991, Immunol. Today 12:51-54; Segal, et al., 1991, Ann. N.Y. Acad. Sci. 636:288-294; Segal et al., 1992, Immunobiology 185:390-402; Wunderlich et al., 1992; Intl. J. Clin. Lab. Res. 22:17-20; George et al., 1994, J. Immunol. 152:1802-1811; Huston et al., 1993, Intl. Rev. Immunol. 10:195-217; Stafford et al., 1993, Cancer Res. 53:4026-4034; Haber et al., 1992, Ann. N.Y. Acad. Sci. 667:365-381; Haber, 1992, Ann. N.Y. Acad. Sci. 667: 365-381; Feloner and Rhodes, 1991, Nature 349:351-352; Sarver and Rossi, 1993, AIDS Research & Human Retroviruses 9:483-487; Levine and Friedmann, 1993, Am. J. Dis. Child 147:1167-1176; Friedmann, 1993, Mol. Genetic Med. 3:1-32; Gilboa and Smith, 1994, Trends in Genetics 10:139-144; Saito et al., 1994, Cancer Res. 54:3516-3520; Li et al., 1994, Blood 83:3403-3408; Vieweg et al., 1994, Cancer Res. 54:1760-1765; Lin et al., 1994, Science 265:666-669; Lu et al., 1994, Human Gene Therapy 5:203-208; Gansbacher et al., 1992, Blood 80:2817-2825; Gastl et al., 1992, Cancer Res. 52:6229-6236.

### 2.1 Bacterial Infections and Cancer

Regarding bacteria and cancer, an historical review reveals a number of clinical observations in which cancers were reported to regress in patients with bacterial infections. Nauts et al., 1953, Acta Medica. Scandinavica 145:1-102, (Suppl. 276) state:

The treatment of cancer by injections of bacterial products is based on the fact that for over two hundred years neoplasms have been observed to regress following acute infections, principally streptococcal. If these cases were not too far advanced and the infections were of sufficient severity or duration, the tumors completely disappeared and the patients remained free from recurrence.

Shear, 1950, J. A.M.A. 142:383-390 (Shear), observed that 75 percent of the spontaneous remissions in untreated leukemia in the Children's Hospital in Boston occurred following an acute episode of bacterial infection. Shear questioned:

Are pathogenic and non-pathogenic organisms one of Nature's controls of microscopic foci of malignant disease, and in making progress in the control of infectious diseases, are we removing one of Nature's controls of cancer?

Subsequent evidence from a number of research laboratories indicated that at least some of the anti-cancer effects are mediated through stimulation of the host immune system, resulting in enhanced immuno-rejection of the cancer cells. For example, release of the lipopolysaccharide (LPS) endotoxin by gram-negative bacteria such as Salmonella triggers release of tumor necrosis factor, TNF, by cells of the host immune system, such as macrophages, Christ et al., 1995, Science 268:80-83. Elevated TNF levels in turn initiate a cascade of cytokine-mediated reactions which culminate in the death of tumor cells. In this regard, Carswell et al., 1975, Proc. Natl. Acad. Sci. USA 72:3666-3669, demonstrated that mice injected with bacillus Calmette-Guerin (BCG) have increased serum levels of TNF and that TNF-positive serum caused necrosis of the sarcoma Meth A and other transplanted tumors in mice. Further, Klimpel et al., 1990, J. Immunol. 145:711-717, showed that fibroblasts infected in vitro with Shigella or Salmonella had increased susceptibility to TNF.

As a result of such observations as described above, immunization of cancer patients with BCG injections is currently utilized in some cancer therapy protocols. See Sosnowski, 1994, Compr. Ther. 20:695-701; Barth and Morton, 1995, Cancer 75 (Suppl. 2):726-734; Friberg, 1993, Med. Oncol. Tumor. Pharmacother. 10:31-36 for reviews of BCG therapy.

### 2.2 Parasites and Cancer Cells

Although the natural biospecificity and evolutionary adaptability of parasites has been recognized for some time and the use of their specialized systems as models for new therapeutic procedures has been suggested, there are few reports of, or proposals for, the actual use of parasites as vectors.

Lee et al., 1992, Proc. Natl. Acad. Sci. USA 89:1847-1851 (Lee et al.) and Jones et al, 1992, Infect. Immun. 60:2475-2480 (Jones et al.) isolated mutants of Salmonella typhimurium that were able to invade HEp-2 (human epidermoid carcinoma) cells in vitro in significantly greater numbers than the wild type strain. The “hyperinvasive” mutants were isolated under conditions of aerobic growth of the bacteria that normally repress the ability of wild type strains to invade HEp-2 animal cells. However, Lee et al. and Jones et al did not suggest the use of such mutants as therapeutic vectors, nor did they suggest the isolation of tumor-specific bacteria by selecting for mutants that show infection preference for melanoma or other cancers over normal cells of the body. Without tumor-specificity or other forms of attenuation, such hyperinvasive Salmonella typhimurium as described by Lee et al. and Jones et al. would likely be pan-invasive, causing wide-spread infection in the cancer patient.

### 2.3 Tumor-Targeted Bacteria

Genetically engineered Salmonella have been demonstrated to be capable of tumor targeting, possess anti-tumor activity and are useful in delivering effector genes such as the herpes simplex thymidine kinase (HSV TK) to solid tumors (Pawelek et al., WO 96/40238). Two significant considerations for the in vivo use of bacteria are their virulence and ability to induce tumor necrosis factor α (TNFα)-mediated septic shock. As TNFα-mediated septic shock is among the primary concerns associated with bacteria, modifications which reduce this form of an immune response would be useful because TNFα levels would not become toxic, and a more effective concentration and/or duration of the therapeutic vector could be used.

### 2.4 Modified Bacterial Lipid A

Modifications to the lipid composition of tumor-targeted bacteria which alter the immune response as a result of decreased induction of TNFα production were suggested by Pawelek et al. (Pawelek et al., WO 96/40238). Pawelek et al. provided methods for isolation of genes from Rhodobacter responsible for monophosphoryl lipid A (MLA) production. MLA acts as an antagonist to septic shock. Pawelek et al. also suggested the use of genetic modifications in the lipid A biosynthetic pathway, including the mutation firA, which codes for the third enzyme UDP-3-O (R-30 hydroxylmyristoly)-glucosamine N-acyltransferase in lipid A biosynthesis (Kelley et al., 1993, J. Biol. Chem. 268: 19866-19874). Pawelek et al. showed that mutations in the firA gene induce lower levels of TNFα. However, these authors did not suggest enzymes which modify the myristate portion of the lipid A molecule. Furthermore, Pawelek et al. did not suggest that modifications to the lipid content of bacteria would alter their sensitivity to certain agents, such as chelating agents.

In Escherichia coli, the gene msbB (mlt) which is responsible for the terminal myristalization of lipid A has been identified (Engel, et al., 1992, J. Bacteriol. 174:6394-6403; Karow and Georgopoulos 1992, J. Bacteriol. 174: 702-710; Somerville et al., 1996, J. Clin. Invest. 97: 359-365). Genetic disruption of this gene results in a stable non-conditional mutation which lowers TNFα induction (Somerville et al., 1996, J. Clin. Invest. 97: 359-365; Somerville, WO 97/25061). These references, however, do not suggest that disruption of the msbB gene in tumor-targeted Salmonella vectors would result in bacteria which are less virulent and more sensitive to chelating agents.

The problems associated with the use of bacteria as gene delivery vectors center on the general ability of bacteria to directly kill normal mammalian cells as well as their ability to overstimulate the immune system via TNFα which can have toxic consequences for the host (Bone, 1992, JAMA 268: 3452-3455; Dinarello et al., 1993, JAMA 269: 1829-1835). In addition to these factors, resistance to antibiotics can severely complicate coping with the presence of bacteria within the human body (Tschape, 1996, D T W Dtsch Tierarztl Wochenschr 1996 103:273-7; Ramos et al., 1996, Enferm Infec. Microbiol. Clin. 14: 345-51).

Hone and Powell, WO97/18837 (“Hone and Powell”), disclose methods to produce gram-negative bacteria having non-pyrogenic Lipid A or LPS. Although Hone and Powell broadly asserts that conditional mutations in a large number of genes including msbB, kdsA, kdsB, kdtA, and htrB, etc. can be introduced into a broad variety of gram-negative bacteria including E. coli, Shigella sp., Salmonella sp., etc., the only mutation exemplified is an htrB mutation introduced into E. coli. Further, although Hone and Powell propose the therapeutic use of non-pyrogenic Salmonella with a mutation in the msbB gene, there is no enabling description of how to accomplish such use. Moreover, Hone and Powell propose using non-pyrogenic bacteria only for vaccine purposes.

Maskell, WO98;/33923, describes a mutant strain of Salmonella having a mutation in the msbB gene which induces TNFα at a lower level as compared to a wild type strain.

Bermudes et al, WO 99/13053, teach compositions and methods for the genetic disruption of the msbB gene in Salmonella, which results in Salmonella possessing a lesser ability to elicit TNFα and reduced virulence compared to the wild type. In certain embodiments, some such mutant Salmonella have increased sensitivity to chelating agents as compared to wild type Salmonella.

The preferred properties of tumor-specific Salmonella strains include 1) serum resistance, allowing the parasite to pass through the vasculature and lymphatic system in the process of seeking tumors, 2) facultative anaerobiasis, i.e., ability to grow under anaerobic or aerobic conditions allowing amplification in large necrotic tumors which are hypoxic as well as small metastatic tumors which may be more aerobic, 3) susceptibility to the host's defensive capabilities, limiting replication in normal tissues but not within tumors where the host defensive capabilities may be impaired, 4) attenuation of virulence, whereby susceptibility to the host defenses may be increased, and the parasite is tolerated by the host, but does not limit intratumoral replication, 5) invasive capacity towards tumor cells, aiding in tumor targeting and anti-tumor activity, 6) motility, aiding in permeation throughout the tumor, 7) antibiotic sensitivity for control during treatment and for post treatment elimination (e.g., sensitivity to ampicillin, chloramphenicol, gentamicin, ciprofloxacin), and lacking antibiotic resistance markers such as those used in strain construction, and 8) low reversion rates of phenotypes aiding in the safety to the recipient individual.

### 2.5 Filamentous Phage

Bacteriophages, such as lambda and filamentous phage, have occasionally been used to transfer DNA into mammalian cells. In general, transduction of lambda was found to be a relatively rare event and the expression of the reporter gene was weak. In an effort to enhance transduction efficiency, methods utilizing calcium phosphate or liposomes were used in conjunction with lambda. Gene transfer has been observed via lambda using calcium phosphate co-precipitation (Ishiura et al., 1982, Mol. Cell. Biol. 2:607-616) or via filamentous phage using DEAE-dextran or lipopolyamine (Yokoyama-Kobayashi and Kato, 1993, Biochem. Biophys. Res. Comm. 192:935-939; Yokoyama-Kobayashi and Kato, 1994, Anal. Biochem. 223:130-134). However, these methods of introducing DNA into mammalian cells are not practical for gene therapy applications, as the transfection efficiency tends to be low. More reliable means of targeting vectors to specific cells and of guaranteeing a therapeutically useful degree of gene delivery and expression are thus required, if the bacteriophage are to be useful in therapeutic applications. One such means is described in International Publication WO 99/10014, which teaches phage particles expressing cell receptor ligands as fusion proteins with the phage capsid proteins.

Citation or identification of any reference in Section 2, or any section of this application shall not be construed as an admission that such reference is available as prior art to the present invention.

### 3. SUMMARY OF THE INVENTION

The present invention provides a means to deliver a nucleic acid molecule which encodes for a gene product useful for treating or preventing one or more of a variety of diseases and disorders. In one embodiment, the gene product is useful to treat or prevent sarcomas, carcinomas, or other solid tumor cancers. In another embodiment, the gene product is useful for inducing an immune response to an antigen which is either encoded by, or is expressed on the surface of, a bacteriophage of the present invention.

The present invention is directed to attenuated and/or tumor targeting bacteria, such as Salmonella spp., which contain a filamentous bacteriophage, wherein the genome of the bacteriophage has been modified to encode for a gene product of interest under the control of an appropriate eukaryotic promoter or wherein the genome of the bacteriophage has been modified to encode for a gene product of interest as a fusion protein with a bacteriophage capsid protein, e.g., phage protein III or VIII. The gene product of interest is a proteinaceous molecule, e.g., protein, peptide, glycosylated protein, or is a nucleic acid molecule. Optionally, the attenuated bacteria is able to selectively target and/or invade a solid tumor. Also optionally, the attenuated, tumor-targeting bacteria can be modified to express the F′ pilus. In another embodiment, the genome of the filamentous bacteriophage has also been modified to express an endosomal escape moiety, preferably as a gene fusion with a phage capsid protein, such as capsid protein III or VIII of filamentous phage M13 or fl. In yet another embodiment, the gene product of interest is expressed as a gene fusion with a ferry protein to enhance the internalization of the expressed gene product into a tumor cell. This embodiment is particularly advantageous if not all attenuated phage-containing bacteria are internalized in a tumor cell of a solid tumor but rather are located in the interstitial spaces of the solid tumor.

The present invention is also directed to attenuated and/or tumor-targeting bacteria which express the F′ pilus enabling such bacteria to be infected by filamentous bacteriophage.

While the teachings of the following sections are discussed, for simplicity, with reference specifically to Salmonella, the compositions and methods of the invention are in no way meant to be restricted to Salmonella but encompass any other Gram-negative bacteria to which the teachings apply. Specifically, the invention provides an attenuated tumor-targeting Gram-negative bacterium which is a facultative aerobe or facultative anaerobe. More specifically, the attenuated tumor-targeting bacteria is selected from the group consisting of: Escherichia coli including enteroinvasive Escherichia coli, Salmonella spp., Shigella spp., Yersinia enterocohtica, and Mycoplasma hominis.

The present invention is directed to methods for the production of non-pyrogenic preparations of filamentous bacteriophage comprising infecting attenuated, Gram-negative bacteria which express the F′ pilus and a modified substituent of the bacterium that allows for the elimination or mitigation of toxic effects caused by the wild-type substituent, culturing the infected bacteria under conditions allowing for bacterial growth and phage production and collecting the produced phage from the bacterial culture. In a specific embodiment, the present invention is directed to methods for the production of non-pyrogenic preparations of bacteriophage comprising infecting attenuated msbB⁻ Salmonella expressing the F′ pilus, culturing the infected Salmonella under conditions allowing for bacterial growth and phage production and collecting the produced phage from the Salmonella culture.

The present invention is also directed to a method for delivering an agent for treating or preventing a disease or disorder comprising administering, to a subject in need of such treatment or prevention, a pharmaceutical composition comprising an attenuated Salmonella containing a bacteriophage, wherein the bacteriophage genome has been modified to encode for a gene product of interest under the control of an appropriate eukaryotic promoter or wherein the genome of the bacteriophage has been modified to encode for a gene product of interest as a fusion protein with a bacteriophage capsid protein. The present invention is also directed to a method for inducing an immune response to an antigen comprising administering, to a subject, a pharmaceutical composition comprising an attenuated Salmonella containing a bacteriophage, wherein the genome of the bacteriophage has been modified to encode for an antigen under the control of an appropriate eukaryotic promoter or wherein the genorne of the bacteriophage has been modified to encode for an antigen as a fusion with a bacteriophage capsid protein, e.g., capsid protein III or VIII. In certain aspects of this embodiment, the antigen is a tumor-associated antigen.

The present invention is also directed to a method of treating solid tumors comprising administering, to et subject in need of such treatment, a pharmaceutical composition comprising an attenuated, tumor-targeting Salmonella containing a bacteriophage, wherein the bacteriophage genome has been modified to encode for a gene product of interest under the control of an appropriate eukaryotic promoter or wherein the genome of the bacteriophage is modified to encode for a gene product of interest as a fusion protein with a bacteriophage capsid protein. Solid tumors include, but are not limited to, sarcomas, carcinomas and other solid tumor cancers, such as germ line tumors and tumors of the central nervous system. including, but not limited to, breast cancer, prostate cancer, cervical cancer, uterine cancer, lung cancer, ovarian cancer, testicular cancer, thyroid cancer, astrocytoma, glioma, mesothelioma, bladder cancer, renal cancer, pancreatic cancer, stomach cancer, liver cancer, colon cancer, and melanoma.

In a preferred embodiment, the bacteriophage genome is a phagemid.

The gene product of interest is selected from the group consisting of proteinaceous and nucleic acid molecules. In various embodiments, the proteinaceous molecule is a cellular toxin (cytotoxic agent), e.g., saporin, cytotoxic necrotic factor-1 or cytotoxic necrotic factor-2, a ribosome inactivating protein, or a porin protein, such as gonococcal PI porin protein. In other embodiments, the proteinaceous molecule is an anti-angiogenesis protein or an antibody. In yet other embodiments, the proteinaceous molecule is a cytokine, e.g., IL-2, or a pro-drug converting enzyme, e.g., Herpes Simplex Virus (“HSV”) thymidine kinase or cytosine deaminase. The nucleic acid molecule can be double stranded or single stranded DNA or double stranded or single stranded RNA, as well as triplex nucleic acid molecules. The nucleic acid molecule can function as a ribozyme, DNazyme or antisense nucleic acid, etc.

In a particular embodiment, the gene product of interest comprises a number of viral gene products. For example, the gene product of interest comprises all the viral proteins encoded by an adenovirus or herpesvirus or reovirus genome. In a particular example, the gene product of interest is all the viral proteins encoded by an adenovirus genome except for the E1B viral protein such that this particular adenovirus can only replicate in a mammalian cell lacking p53 activity. Hence in this case the phage genome contains a nucleic acid encoding for all of the adenovirus genome except for E1B and a phage origin of replication. In this particular case wherein the Salmonella containing phage are administered to an organism and delivered to a tumor cell, the produced adenovirus can only replicate in a cell lacking p53 activity, i.e., another tumor cell.

The present invention is also directed to a pharmaceutical composition comprising a pharmaceutically acceptable carrier and an attenuated Salmonella containing a bacteriophage, wherein the genome of the bacteriophage has been modified to encode for a gene product of interest under the control of an appropriate eukaryotic promoter or wherein the genome of the bacteriophage has been modified to encode for a gene product of interest as a fusion protein with a bacteriophage capsid protein. Optionally, the attenuated Salmonella strain is also a tumor-targeting strain.

### 3.1. Definitions

As used herein, Salmonella encompasses all Salmonella species, including: Salmonella typhi, Salmonella choleraesuis, and Salmonella enteritidis. Serotypes of Salmonella are also encompassed herein, for example, typhimurium, a subgroup of Salmonella enteritidis, commonly referred to as Salmonella typhimurium.

Anti-angiogenic factor: An anti-angiogenic factor is any proteinaceous molecule which has anti-angiogenic activity, or a nucleic acid encoding such a proteinaceous molecule. In a preferred embodiment, the anti-angiogenic factor is a peptide fragment or cleavage fragment of a larger protein.

Attenuation: Attenuation is a modification so that a microorganism or vector is less pathogenic. The end result of attenuation is that the risk of toxicity as well as other side-effects is decreased, when the microorganism or vector is administered to the patient.

Bacteriocin: As used herein, a bacteriocin is a bacterial proteinaceous toxin with selective activity, in that its bacterial host is immune to the toxin. Bacteriocins may be encoded by the bacterial genome or by a plasmid, may be toxic to a broad or narrow range of other bacteria, and may have a simple structure comprising one or two subunits or may have a multi-subunit structure. In addition, a host expressing bacteriocin has immunity against the bacteriocin.

Cytotoxin: As used herein, cytotoxin refers to a compound that results in cell death or cell stasis occurring through apoptosis, necrosis or other mechanism.

Virulence: Virulence is a relative term describing the general ability to cause disease, including the ability to kill normal cells or the ability to elicit septic shock (see specific definition below).

Septic shock: Septic shock is a state of internal organ failure due to a complex cytokine cascade, initiated by TNFα. The relative ability of a microorganism or vector to elicit TNFα is used as one measure to indicate its relative ability to induce septic shock.

Gene product: Gene product refers to any molecule capable of being encoded by a nucleic acid, including but not limited to, a protein or another nucleic acid, e.g., DNA, RNA dsRNAi, ribozyme, DNazyme, etc. The nucleic acid which encodes for the gene product of interest is not limited to a naturally occurring full-length “gene” having non-coding regulatory elements.

Tumor targeting: Tumor targeting is defined as the ability to distinguish between a cancerous target cell and the non-cancerous counterpart cell or tumor tissue from non-tumor tissue so that a tumor targeting Salmonella preferentially attaches to, infects and/or remains viable in the cancerous target cell or the tumor environment.

Chelating agent sensitivity: Chelating agent sensitivity is defined as the effective concentration at which bacteria proliferation is affected, or the concentration at which the viability of bacteria, as determined by recoverable colony forming units (c.f.u.), is reduced.

As used herein, the strain designations VNP20009 (International Publication No. WO 99/13053), YS 1646 and 41.2.9 are used interchangeably and each refer to the strain deposited with the American Type Culture Collection and assigned Accession No. 202165. As used herein, the strain designations YS1456 and 8.7 are used interchangeably and each refer to the strain deposited with the American Type Culture Collection and assigned Accession No. 202164.

### 3.2 OBJECTS OF THE INVENTION

One object of the present invention is to provide a vector for delivering a gene product or a nucleic acid which encodes a gene product of interest into a mammalian or avian cell. Another object of the invention is to provide a vector for delivering a gene product or a nucleic acid which encodes a gene product of interest to the site of a solid tumor or into a solid tumor cell. Yet another object of the invention is to provide methods for the treatment or prevention of disease and disorders, including solid tumor cancers, using the vectors of the present invention.

The present invention may be understood more fully by reference to the following detailed description, illustrative examples of specific embodiments and the appended figures.

### 4. BRIEF DESCRIPTION OF THE FIGURES

FIGS. 1A and 1B show representative photographs of transient transfection and expression of green fluorescent protein (“GFP”) expressed from single stranded phagemid DNA (PBSKIIGFP) isolated from Salmonella.

FIGS. 2A-2B FIG. 2A is representative photograph of delivery, transfection and expression of GFP in a mammalian cell line, COS 7, using live Salmonella as the carrier. FIG. 2B is a representative photograph of a control cell not transfected with live Salmonella.

FIGS. 3A-3B. FIGS. 3A-3B are Western blots demonstrating expression of an IL-2/pIII fusion protein. FIG. 3A is a blot probed with an anti-IL-2 antibody and FIG. 3B is a blot probed with anti-pIII antibody.

FIGS. 4A-4D are graphical representations demonstrating that phage containing the IL-2/pIII fusion protein expressed in two different Salmonella strains has IL-2 activity as measured in a proliferation assay using a IL-2-dependent mouse cytotoxic T cell line, CTLL-2, which activity is concentration dependent. FIGS. 4A-4B show concentration-dependent IL-2 activity from phage expressed in strain VNP20009. FIGS. 4C-4D show concentration-de(pendent IL-2 activity from phage expressed in strain YS1456.

### 5. DETAILED DESCRIPTION OF THE INVENTION

The present invention provides a means to deliver a nucleic acid molecule which encodes a gene product useful for treating or preventing various diseases and disorders. As used herein, the term treatment encompasses inhibition of progression of symptoms or amelioration of symptoms of a disease or disorder. In one embodiment, the gene product is useful to treat or prevent sarcomas, carcinomas, or other solid tumor cancers. In another embodiment, the gene product is useful for inducing an immune response to an antigen which is either encoded by, or is expressed on the surface of, a bacteriophage of the present invention. The immune response can be directed against a tumor or an infectious agent.

Although not intending to be limited to any one mechanism, the inventors believe that certain embodiments of the present invention result in the targeted expression of the encoded gene product of interest by delivery of the phage to a cell by endocytosis of the attenuated bacterial vector containing the phage into a cell endosome, replication of the phage in the bacteria and secretion of the phage into the endosome, escape of the phage from the endosome into the cytoplasm, and translocation to the nucleus wherein the phage-encoded gene product of interest is expressed. Another non-limiting mechanism by which the present invention is believed to operate in certain embodiments is secretion of the phage by the bacteria into the interstitial space of a solid tumor and the subsequent uptake of the phage by the tumor cells, wherein expression of the gene product of interest occurs. Yet another non-limiting mechanism by which the present invention is believed to operate in certain embodiments is not all attenuated phage-containing bacteria are internalized in a tumor cell of a solid tumor but rather are located in the interstitial spaces of the solid tumor and the expressed gene product of interest is internalized into a tumor cell.

As indicated above, bacterial vectors, according to the present invention are attenuated and contain a bacteriophage, wherein the genome of the bacteriophage has been modified to encode for a gene product of interest under the control of an appropriate eukaryotic promoter or wherein the genome of the bacteriophage has been modified to encode for a gene product of interest as a fusion protein with a bacteriophage capsid protein, e.g., phage capsid protein III or VIII. For reasons of clarity, the detailed description is divided into the following subsections: (1): Bacterial Vectors; (2): Filamentous Phage; (3): Gene Product of Interest; and (4): Methods and Compositions for Delivery of an Agent.

### 5.1 Bacterial Vectors

Any attenuated tumor-targeting Gram-negative bacteria may be used in the methods of the invention. More specifically, the attenuated tumor-targeted bacteria are facultative aerobes or facultative anaerobes and are selected from the group consisting of: Escherichia coli, enteroinvasive Escherichia coli, Salmonella spp., Shigella spp., Yersinia enterocohtica, and Mycoplasma hominis.

While the teachings of the following section refers specifically to Salmonella, the compositions and methods of the invention are in no way meant to be restricted to Salmonella but encompass any other Gram-negative bacterium to which the teachings apply. Suitable bacterial species include, but are not limited to, Escherichia coli, enteroinvasive Escherichia coli, Salmonella spp., Shigella spp., Yersinia enterocohtica, and Mycoplasma hominis, wherein the bacterium is a Gram-negative facultative aerobe or facultative anaerobe.

Salmonella is a causative agent of disease in humans and animals. One such disease that can be caused by Salmonella is sepsis, which is a serious problem because of the high mortality rate associated with the onset of septic shock (R. C. Bone, 1993, Clinical Microbiol. Revs. 6:57-68). Therefore, to allow the safe use of Salmonella vectors in the present invention, the Salmonella vectors are attenuated in their virulence for causing disease. In the present application, attenuation, in addition to its traditional definition in which a microorganism vector is modified so that the microorganism vector is less pathogenic, is intended to include also the modification of a microorganism vector so that a lower titer of that derived microorganism vector can be administered to a patient and still achieve comparable results as if one had administered a higher titer of the parental microorganism vector. The end result is to reduce the risk of septic shock or other side effects due to administration of the vector to the patient. Such attenuated microorganisms are isolated by means of a number of techniques.

Suitable methods for obtaining attenuated Salmonella include use of antibiotic-sensitive strains of microorganisms, mutagenesis of the microorganisms, selection for tumor-specific and/or super-infective microorganism mutants in culture or in tumor-bearing animals, selection for microorganism mutants that lack virulence factors necessary for survival in normal cells, including macrophages and neutrophils, and construction of new strains of microorganisms with altered cell wall lipopolysaccharides. For example, Section 6.1 of International Publication WO 96/40238, which publication is incorporated by reference in its entirety herein, describes methods for the isolation of tumor-targeting Salmonella vectors, these same methods are also methods for isolating attenuated vectors; super-infective and/or tumor-targeting Salmonella vectors are by definition attenuated. However, not all attenuated Salmonella vectors are tumor-targeting. As the vectors are highly specific and super-infective, the difference between the number of infecting Salmonella found at the target tumor cell as compared to the non-cancerous counterparts becomes larger and larger as the dilution of the microorganism culture is increased such that lower titers of microorganism vectors can be used with positive results. Thus, in a preferred embodiment of the present invention, the Salmonella vector is a tumor-targeting strain of Salmonella.

Further, the Salmonella can be attenuated by the deletion or disruption of DNA sequences which encode for virulence factors which insure survival of the Salmonella in the host cell, especially macrophages and neutrophils, by, for example, homologous recombination techniques and chemical or transposon mutagenesis. For example, a number of these virulence factors have been identified in Salmonella. Many, but not all, of these studied virulence factors are associated with survival in macrophages such that these factors are specifically expressed within macrophages due to stress, for example, acidification, or are used to induce specific host cell responses, for example, macropinocytosis (Fields et al., 1986, Proc. Natl. Acad. Sci. USA 83:5189-5193). Table 4 of International Publication WO 96/40238 is an illustrative list of Salmonella virulence factors which, if deleted by homologous recombination techniques or chemical or transposon mutagenesis, result in attenuated Salmonella.

Yet another method for the attenuation of the Salmonella vectors is to modify substituents of the microorganism which are responsible for the toxicity of that microorganism. For example, lipopolysaccharide (LPS) or endotoxin is primarily responsible for the pathological effects of bacterial sepsis. The component of LPS which results in this response is lipid A (“LA”). Elimination or mitigation of the toxic effects of LA results in an attenuated bacteria since 1) the risk of septic shock in the patient would be reduced and 2) higher levels of the bacterial vector could be tolerated.

As an illustrative example, the generation of mutant LA producing Salmonella entails constructing a DNA gene library composed of 10 kB fragments from an organism that expresses mutant LA, e.g., Rhodobacter sphaeroides, which is generated in λgtll or pUC19 plasmids and transfected into Salmonella. Clones which produce mutant LA are positively selected by using an antibody screening methodology to detect mutant LA, such as ELISA. In another example one generates a cosmid library composed of 40 kB DNA fragments from an organism that expresses mutant LA in pSuperCos which is then transfected into Salmonella. Clones which produce mutant LA are positively selected by using an antibody screening methodology to detect mutant LA, such as ELISA.

Yet another example for altering the LPS of Salmonella involves the introduction of mutations in the LPS biosynthetic pathway. Several enzymatic steps in LPS biosynthesis and the genetic loci controlling them in Salmonella have been identified (Raetz, 1993, J. Bacteriol. 175:5745-5753 and references therein). Several mutant strains of Salmonella have been isolated with genetic and enzymatic lesions in the LPS pathway. One such illustrative mutant, firA is a mutation within the gene that encodes the enzyme UDP-3-O(R-30 hydroxymyristoyl)-glycocyamine N-acyltransferase, that regulates the third step in endotoxin biosynthesis (Kelley et al., 1993, J. Biol. Chem. 268:19866-19874). Salmonella strains bearing this type of mutation produce a lipid A that differs from wild type lipid A in that it contains a seventh fatty acid, a hexadecanoic acid (Roy and Coleman, 1994, J. Bacteriol. 176:1639-1646). Roy and Coleman demonstrated that in addition to blocking the third step in endotoxin biosynthesis, the firA⁻ mutation also decreases enzymatic activity of lipid A 4′ kinase that regulates the sixth step of lipid A biosynthesis.

Another illustrative example of such a LPS pathway mutant is the msbB⁻ mutant described in International Publication WO 99/13053, which publication is incorporated herein by reference. One characteristic of the msbB⁻ Salmonella is decreased ability to induce a TNFα response compared to the wild type bacterial vector. The msbB⁻ Salmonella induce TNFα expression at about 5 percent to about 40 percent compared to the wild type Salmonella sp. (in other words, the msbB⁻ Salmonella induce TNFα expression at about 5 percent to about 40 percent of the level induced by wild type Salmonella). In a preferred embodiment, the present invention encompasses a msbB⁻ Salmonella vector that induces TNFα expression at about 10 percent to about 35 percent of that induced by a wild type Salmonella and contains a bacteriophage, wherein the genome of the bacteriophage encodes for an agent under the control of an eukaryotic promoter. In another embodiment, the invention encompasses a mutant msbB⁻ Salmonella vector which produces LPS which when purified induces TNFα expression at a level which is less than or equal to 0.001 percent of the level induced by LPS purified from wild type Salmonella sp. TNFα response induced by whole bacteria or isolated or purified LPS can be assessed in vitro or in vivo using commercially available assay systems such as by enzyme linked immunoassay (ELISA). Comparison of TNFα production on a per colony forming unit (“c.f.u.”) or on a μg/kg basis, is used to determine relative activity. Lower TNFα levels on a per unit basis indicate decreased induction of TNFα production.

Another characteristic of the msbB⁻ Salmonella vector is decreased virulence towards the patient compared to the wild type bacterial vector. Wild type Salmonella can under some circumstances exhibit the ability to cause significant progressive disease. Acute lethality can be determined for normal wild type live Salmonella and live msbB⁻ Salmonella using animal models. Comparison of animal survival for a fixed inoculum is used to determine relative virulence. Strains having a higher rate of survival of animal host have decreased virulence.

Another characteristic of msbB⁻ Salmonella is decreased survival within macrophage cells as compared to survival of wild type bacteria. Wild type Salmonella are noted for their ability to survive within macrophages (Baumler, et al., 1994, Infect. Immun. 62:1623-1630; Buchmeier and Heffron 1989, Infect. Immun. 57:1-7; Buchmeier and Heffron, 1990, Science 248:730-732; Buchmeier et al., 1993, Mol. Microbiol. 7:933-936; Fields et al., 1986, Proc. Natl. Acad. Sci. USA 83:5189-93; Fields et al., 1989, Science 243:1059-62; Fierer et al., 1993, Infect. Immun. 61:5231-5236; Lindgren et al., 1996, Proc. Natal. Acad. Sci. USA 3197-4201; Miller et al., 1989, Proc. Natl. Acad. Sci. USA 86:5054-5058; Sizemore et al., 1997, Infect. Immun. 65:309-312).

A comparison of survival time in macrophages can be made using an in vitro cell culture assay, as described in International Publication WO 99/13053. A lower number of colony forming units (“c.f.u.”) over time is indicative of reduced survival within macrophages. In an embodiment of the invention, survival of msbB⁻ Salmonella occurs at about 50 percent to about 30 percent; preferably at about 30 percent to about 10 percent; more preferably at about 10 percent to about 1 percent of survival of the wild type stain.

Another characteristic of one embodiment of the msbB⁻ Salmonella is increased sensitivity of the bacteria to specific chemical agents which is advantageously useful to assist in the elimination of the bacteria after administration in vivo. Bacteria are susceptible to a wide range of antibiotic classes. However, WO 99/13053 teaches that certain Salmonella msbB⁻ mutants are more sensitive to certain chemicals which are not normally considered antibacterial agents. In particular, certain msbB⁻ Salmonella mutants are more sensitive than wild type Salmonella to chelating agents.

To determine sensitivity to chemical agents, normal wild type Salmonella and msbB⁻ Salmonella are compared for growth in the presence or absence of a chelating agent, for example, EDTA, EGTA or sodium citrate. Comparison of growth is measured as a function of optical density, i.e., a lower optical density in the msbB⁻ strain grown in the presence of an agent, than when the strain is grown in its absence, indicates sensitivity. Furthermore, a lower optical density in the msbB⁻ strain grown in the presence of an agent, compared to the msbB⁺ strain grown in the presence of the same agent, indicates sensitivity specifically due to the msbB mutation. In an embodiment of the invention, 90 percent inhibition of growth of msbB⁻ Salmonella (compared to growth of wild type Salmonella) occurs at about 0.25 mM EDTA to about 0.5 mM EDTA, preferably at about 99 percent inhibition at about 0.25 mM EDTA to above 0.5 mM EDTA, more preferably at greater than 99 percent inhibition at about 0.25 mM EDTA to about 0.5 mM EDTA. Similar range of growth inhibition is observed at similar concentrations of EGTA.

The present invention also encompasses the use of derivatives of msbB⁻ attenuated mutants. When grown in Luria Broth (LB) containing zero salt, the msbB⁻ mutants of the present invention are stable, i.e., produce few derivatives. Continued growth of the msbB⁻ mutants on modified LB (10 g tryptone, 5 g yeast extract, 2 ml 1N CaCl₂, and 2 ml 1N MgSO₄ per liter, adjusted to pH 7 using 1N NaOH) also maintains stable mutants.

In contrast, when grown in normal LB, the msbB⁻ mutants may give rise to derivatives. As used herein, “derivatives” is intended to mean spontaneous variants of the msbB⁻ mutants characterized by a different level of virulence, tumor inhibitory activity and/or sensitivity to a chelating agent when compared to the original msbB⁻ mutant. The level of virulence, tumor inhibitory activity, and sensitivity to a chelating agent of a derivative may be greater, equivalent, or less compared to the original msbB⁻ mutant.

Derivatives of msbB⁻ strains grow faster on unmodified LB than the original msbB⁻ strains. In addition, derivatives can be recognized by their ability to grow on MacConkey agar (an agar which contains bile salts) and by their resistance to chelating agents, such as EGTA and EDTA. Derivatives can be stably preserved by cryopreservation at −70° C. or lyophilization according to methods well known in the art (Cryz et al., 1990, In New Generation Vaccines, M. M. Levine (ed.), Marcel Dekker, New York pp. 921-932; Adams, 1996, In Methods in Molecular Medicine: Vaccine Protocols, Robinson et al. (eds), Humana Press, New Jersey, pp. 167-185; Griffiths, Id. pp. 269-288.)

Virulence is determined by evaluation of the administered dose at which half of the animals die (LD₅₀). Comparison of the LD₅₀ of the derivatives can be used to assess the comparative virulence. Decrease in the LD₅₀ of a spontaneous derivative as compared to its msbB⁻ parent, indicates an increase in virulence. In an illustrative example, the faster-growing derivatives either exhibit the same level of virulence, a greater level of virulence, or a lower level of virulence compared to their respective original mutant strains. In another example, the ability of a derivative to induce TNFα remains the same as the original mutant strain. In an illustrative example, the derivatives can either inhibit tumor growth more than or less than their respective original mutant strains.

A derivative which is more virulent than its parent mutant but which does induce TNFα at a lower level when compared to the wild type, i.e., at a level of about 5 percent to about 40 percent of that induced by the wild type Salmonella, can be further modified to contain one or more mutations to auxotrophy. In an illustrative example, a msbB⁻ derivative is mutated such that it is auxotrophic for one or more aromatic amino acids, e.g., aroA, and thus can be made less virulent and is useful according to the methods of the present invention. In an additional illustrative example, genetic modifications of the purI gene (involved in purine biosynthesis) yield Salmonella strains that are less virulent than the parent strain.

Prior to use of a derivative in the methods of the invention, the derivative is assessed to determine its level of virulence, ability to induce TNFα, ability to inhibit tumor growth, and sensitivity to a chelating agent.

Once the Salmonella strain has been attenuated by any of the methods known in the art, the stability of the attenuated phenotype is important such that the strain does not revert to a more virulent phenotype during the course of treatment of a patient. Such stability can be obtained, for example, by providing that the virulence gene is disrupted by deletion or other non-reverting mutations on the chromosomal level rather than epistatically.

Another method of insuring the attenuated phenotype is to engineer the bacteria such that it is attenuated in more than one manner, e.g., a mutation in the pathway for lipid A production, such as the msbB⁻ mutation (International Publication WO 99/13053) and one or more mutations to auxotrophy for one or more nutrients or metabolites, such as uracil biosynthesis, purine biosynthesis, and arginine biosynthesis as described by Bochner, 1980, J. Bacteriol. 143:926-933. In a more preferred embodiment of the invention, the attenuated Salmonella vector also selectively targets tumors. In a yet more preferred embodiment, the Salmonella strain is a tumor-targeting strain, is additionally attenuated by the presence of the msbB⁻ mutation, and is auxotrophic for purine.

Another method is to engineer the Salmonella to be more sensitive to x-rays, ultraviolet radiation, mitomycin or other DNA-damaging agents including free radicals (e.g., oxygen, alkylating agents and nitrogen radicals), oxides, superoxides.

Additionally, since the Salmonella vectors for use in the present invention contain a bacteriophage, the Salmonella strain can also be genetically modified by any method known in the art to express the F′ pilus such that the Salmonella vector can more efficiently take up the bacteriophage. See, generally, Sambrook et al., 1989, Molecular Biology: A Laboratory Manual, Cold Spring Harbor Press, Cold Spring Harbor, N.Y.; Ausubel et al., 1995, Current Protocols in Molecular Biology, Greene Publishing, New York, N.Y. Alternatively, the Salmonella vector can be transfected with phage nucleic acid or phagemid molecules and helper phage.

### 5.2. Filamentous Phage

Filamentous phages encompass a group of bacteriophages that are able to infect a variety of Gram-negative bacteria through interaction with the tip of the F′ pilus. Well known filamentous phages include M13, fl, and fd.

The genomes of these phages are single stranded DNA, but replicate through a double stranded form. Phage particles are assembled in the bacteria and extruded into the media. Because the bacteria continue to grow and divide, albeit at a slower rate than uninfected cells, relatively high titers of phage are obtained. Moreover, replication and assembly appear to be unaffected by the size of the genome. As a consequence of their structure and life cycle, filamentous phages have become a valuable addition in the arsenal of molecular biology tools.

Further development of filamentous phage systems has led to the development of cloning vectors called phagemids, that combine features of plasmids and phages. Phagemids contain an origin of replication and packaging signal of the filamentous phage, as well as a plasmid origin of replication. Other elements that are useful for cloning and/or expression of foreign nucleic acid molecules are generally also present. Such elements include, but are not limited to, selectable genes, multiple cloning sites, and primer sequences. The phagemids may be replicated as for other plasmids and may be packaged into phage particles upon rescue by a helper filamentous phage.

Filamentous phages have also been developed as a system for displaying proteins and peptides on the surface of the phage particle. By insertion of nucleic acid molecules into genes for capsid proteins, fusion proteins are produced that are assembled into the capsid (Smith, 1985, Science 228:1315; U.S. Pat. No. 5,223,409). As a result, the foreign protein or peptide is displayed on the surface of the phage particle. Methods and techniques for phage display are well known in the art. See also, Kay et al., 1996, Phage Display of Peptides and Proteins: A Laboratory Manual, Academic Press, San Diego, Calif.

Filamentous phage generally fall into two categories: phage genome and phagemids, and are collectively referred to as “phage” herein. Either type of phage may be used within the context of the present invention, preferentially phagemids are utilized. Many such commercial phages are available. For example, the pEGFP phage series commercially available from Clontech, Palo Alto, Calif.; M13 mp, pCANTAB 5E phages commercially available from Pharmacia Biotech, Sweden; pBluescript phage commercially available from Stratagene Cloning Systems, La Jolla, Calif. One exemplary useful commercially available phage is pEGFP-N1 which encodes a green fluorescent protein under the control of the CMV immediate-early promoter. This phage also includes a SV40 origin of replication to enhance gene expression by allowing replication of the phage to high copy number in cells also expressing SV40 T antigen.

Other phages are available in the scientific community or may be constructed using methods well known to those of skill in the art. See, e.g., Smith, 1988, in Vectors: A Survey of Molecular Cloning Vectors and their Uses, Rodriquez and Denhardt, eds., Butterworth, Boston, Mass., pp. 61-84; Sambrook et al., 1989, Molecular Biology: A Laboratory Manual, Cold Spring Harbor Press, Cold Spring Harbor, N.Y.; Ausubel et al., 1995, Current Protocols in Molecular Biology, Greene Publishing, New York, N.Y.

At a minimum, for use in the present invention, the phage must be able to accept a cassette containing a, nucleic acid sequence encoding the gene product of interest and a promoter to control expression of the gene product of interest in operative linkage. Any promoter that is active in the cell in which the phage is delivered by means of the attenuated Salmonella can be used. The phage must also have a phage origin of replication and a packaging signal for assembling the phage DNA with the capsid proteins. Additionally, other elements can be incorporated into the phage. For example, a transcription termination sequence, including a polyadenylation sequence, splice donor and acceptor sites. Other elements useful for expression and maintenance of the construct in mammalian cells or other eukaryotic cells can be incorporated into the phage, e.g., eukaryotic origin of replication. Also, since the phages are conveniently produced in bacterial cells, and especially in the present invention in which bacterial cells are used to deliver the phage to the mammalian cell, elements that are necessary for or enhance propagation of the phage in bacteria are incorporated into the phage, e.g., bacterial origin of replication, selectable marker, etc.

In certain embodiments, the phage and/or helper phage are also modified to make the phage and/or helper phage more genetically stable and/or to prevent transmission of the phage and/or helper phage to other bacteria. For example, the helper phage can be cloned without the F1 origin such that the helper phage cannot package itself, or cloned without the RF origin such that the helper phage is dependent upon another origin of replication for production of double-stranded DNA, or both. In addition, the phage or helper phage can be cloned into a plasmid which shows a high degree of genetic stability in Salmonella, such as a colicin plasmid or a balanced lethal plasmid. Further, the phage or helper phage can be cloned onto the bacterial chromosome in order to confer genetic stability. The helper phage cloned into a plasmid having a high degree of genetic stability or cloned onto the bacterial chromosome can also be cloned without the F1 and/or RF origins. See, e.g., Donnenberg and Kaper, 1991, Infect. Immun. 59:4310-4317. In addition, the phage and/or helper phage, can be cloned into a transposon vector for cloning onto the bacterial chromosome. In a particular embodiment, the phage or helper phage is cloned into a colicin plasmid lacking coding sequences for the immunity protein. Such a phage clone will kill any bacterium into which it is introduced which bacterium also lacks the colicin immunity protein (which can be provided in trans), thereby generating a barrier to transmission. (See, Diaz et al., 1994, Mol. Microbio. 13:855-861). In yet another embodiment, the phage and/or helper phage can be cloned without antibiotic resistance markers, thus enabling bacteria infected with such clones to be killed by standard antibiotic therapy.

The promoter that controls the expression of the gene product of interest should be active or activatible in the target cell. The target cell can be, but is not limited to, a mammalian or avian cell. The mammalian cell can be, but is not limited to, human, canine, feline, equine, bovine, porcine, rodent, etc. The choice of promoter will depend on the type of target cell and the degree or type of expression control desired. Promoters that are suitable for use in the present invention include, but are not limited to, constitutive, inducible, tissue-specific, cell type-specific and temporal-specific. Another type of promoter useful in the present invention is an event-specific promoter which is active or up-regulated in response to the occurrence of an event, such as viral infection. For example, the HIV LTR is an event specific promoter. The promoter is inactive unless the tat gene product is present, which occurs upon HIV infection.

Exemplary promoters useful in the present invention include, but are not limited to, the SV40 early promoter region (Bernoist and Chambon, 1981, Nature 290:304-310), the promoter contained in the 3′ long terminal repeat of Rous sarcoma virus (Yamamoto, et al., 1980, Cell 22:787-797), the herpes thymidine kinase promoter (Wagner et al., 1981, Proc. Natl. Acad. Sci. U.S.A. 78:1441-1445), the cytomegalovirus (“CMV”) promoter, the regulatory sequences of the tyrosinase gene which is active in melanoma cells (Siders et al., 1998, Gen. Ther. 5:281-291), the regulatory sequences of the metallothionein gene (Brinster et al., 1982, Nature 296:39-42); plant expression vectors comprising the nopaline synthetase promoter region (Herrera-Estrella et al., Nature 303:209-213) or the cauliflower mosaic virus 35S RNA promoter (Gardner, et al., 1981, Nucl. Acids Res. 9:2871), and the promoter of the photosynthetic enzyme ribulose biphosphate carboxylase (Herrera-Estrella et al., 1984, Nature 310:115-120); promoter elements from yeast or other fungi such as the Gal 4 promoter, the ADC (alcohol dehydrogenase) promoter, PGK (phosphoglycerol kinase) promoter, alkaline phosphatase promoter, and the following animal transcriptional control regions, which exhibit tissue specificity and have been utilized in transgenic animals: elastase I gene control region which is active in pancreatic acinar cells (Swift et al., 1984, Cell 38:639-646; Ornitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region which is active in testicular, breast, lymphoid and mast cells (Leder et al., 1986, Cell 45:485-495), albumin gene control region which is active in liver (Pinkert et al., 1987, Genes and Devel. 1:268-276), alpha-fetoprotein gene control region which is active in liver (Krumlauf et al., 1985, Mol. Cell. Biol. 5:1639-1648; Hammer et al., 1987, Science 235:53-58; alpha 1-antitrypsin gene control region which is active in the liver (Kelsey et al., 1987, Genes and Devel. 1:161-171), beta-globin gene control region which is active in myeloid cells (Mogram et al., 1985, Nature 315:338-340; Kollias et al., 1986, Cell 46:89-94; myelin basic protein gene control region which is active in oligodendrocyte cells in the brain (Readhead et al., 1987, Cell 48:703-712); myosin light chain-2 gene control region which is active in skeletal muscle (Sani, 1985, Nature 314:283-286), prostate specific antigen gene control region which is active in prostate cells, and gonadotropic releasing hormone gene control region which is active in the hypothalamus (Mason et al., 1986, Science 234:1372-1378).

Another exemplary promoter is one that has enhanced activity in the tumor environment; for example, a promoter that is activated by the anaerobic environment of the tumor such as the P1 promoter of the pepT gene. Activation of the P1 promoter is dependent on the FNR transcriptional activator (Strauch et al., 1985, J. Bacteriol. 156:743-751). In a specific embodiment, the P1 promoter is a mutant promoter that is induced at higher levels under anaerobic conditions than the native P1 promoter, such as the pepT200 promoter whose activity in response to anaerobic conditions is induced by CRP-cAMP instead of FNR (Lombardo et al., 1997, J. Bacteriol. 179:1909-1917). In another embodiment, an anaerobicall-induced promoter is used, e.g., the potABCD promoter. potABCD is an operon that is divergently expressed from pepT under anaerobic conditions. The promoter in the pepT gene responsible for this expression has been isolated (Lombardo et al., 1997, J. Bacteriol. 179:1909-1917) and can be used according to the methods of the present invention.

Yet another exemplary promoter is an antibiotic-induced promoter, such as the tet promoter of the Tn10 transposon. In a preferred embodiment, the tet promoter is multimerized, for example, three-fold. Promoter activity would then be induced by administering to a subject who has been treated with the attenuated tumor-targeted bacteria of the invention an appropriate dose of tetracycline. Although the tet inducible expression system was initially described for eukaryotic systems such as Schizosaccharomyces pombe (Faryar and Gatz, 1992, Current Genetics 21:345-349) and mammalian cells (Lang and Feingold, 1996, Gene 168:169-171), recent studies extend its applicability to bacterial cells.

For example, Stieger et al., 1999, Gene 226:243-252) have shown 80-fold induction of the firefly luciferase gene upon tet induction when operably linked to the tet promoter. An advantage of this promoter is that it is induced at very low levels of tetracycline, approximately 1/10th of the dosage required for antibiotic activity.

Other exemplary promoters include the umuC and sulA promoters (Shinagawa et al., 1983, Gene 23:167-174; Schnarr et al., 1991, Biochemie 73:423-431). The sulA promoter includes the ATG of the sulA gene and the following 27 nucleotides as well as 70 nucleotides upstream of the ATG (Cole, 1983, Mol. Gen. Genet. 189:400-404). Therefore, it is useful both in expressing foreign genes and in creating gene fusions for sequences lacking initiating codons. Another exemplary promoter is the IPTG inducible trk promoter (Pharmacia, Piscataway, N.J.).

In addition to the promoter, repressor sequences, negative regulators, or tissue-specific silencers can be inserted to reduce non-specific expression of the gene product of interest. Moreover, multiple repressor elements may be inserted in the promoter region. One type of repressor sequence is an insulator sequence. Illustrative examples of repressor sequences which silence background transcription are found in Dunaway et al., 1997, Mol. Cell Biol. 17:182-129; Gdula et al., 1996, Proc. Natl. Acad. Sci. USA 93:9378-9383; Chan et al., 1996, J. Virol. 70:5312-5328. In certain embodiments, sequences which increase the expression of the gene product of interest can be inserted in the phage, e.g., ribosome binding sites. Expression levels of the transcript or translated product can be assayed by any method known in the art to ascertain which promoter/repressor sequences affect expression.

In preferred embodiments, the phage has an origin of replication suitable for the cell into which it is delivered for expression of the gene product of interest, e.g., for expression of the gene product of interest in a mammalian cell, an origin of replication for mammalian cells can be used, Viral replication systems, such as EBV ori and EBNA gene, SV 40 ori and T antigen, or BPV ori can be utilized in the phages of the present invention for replication of the phage in mammalian cells. Other mammalian replication systems can also be used. The presence of the target cell-responsive origin of replication can allow for an increase in the copy number of the phage.

Also in preferred embodiments, the phage also encodes for a peptide or other moiety that allows for or promotes the escape of the phage from the endosome. Peptide sequences that confer the ability to escape the endosome are particularly preferred. Such sequences are well known in the art and can be readily cloned as a fusion protein with a capsid protein, e.g., protein III or protein VIII of a filamentous phage. Escape sequences that are useful in the present invention include, but are not limited to, a peptide of Pseudomonas exotoxin (Donnelly et al., 1993, Proc. Natl. Acad. Sci. USA 90:3530-3534);

influenza peptides such as the HA peptide and peptides derived therefrom, such as peptide FPI3; Sendai virus fusogenic peptide; the fusogenic sequence from HIV gp1 protein;

Paradaxin fusogenic peptide; and Melittin fusogenic peptide (see International Publication WO96/41606). Two additional illustrative examples of an endosome-disruptive peptide (also called fusogenic peptides) are GLFEAIEGFIENGWEGMIDGGGC (SEQ ID NO: 1) and GLFEAIEGFIENGWEGMIDGWYGC (SEQ ID NO: 2). In particular embodiments in which the gene product of interest is expressed as a fusion with one of the bacteriophage capsid proteins, the endosomal escape peptide is expressed as a fusion with one of the other bacteriophage capsid proteins. In yet other embodiments, the gene product of interest and the endosomal escape peptide are expressed together as a triple fusion peptide with one of the bacteriophage capsid proteins.

Other peptides useful for disrupting endosomes may be identified by various general characteristics. For example, endosome-disrupting peptides are often about 25-30 residues in length, contain an alternating pattern of hydrophobic domains and acidic domains, and at low pH (e.g., pH 5) from amphipathic alpha helicies. Escape peptides can also be selected using a molecular evolution strategy. Briefly, in one strategy, a chemical library of random peptides is engineered into the VIII protein gene of a phage that also carries a detectable, e.g., green fluorescent protein, or selectable, e.g., drug resistance, marker. Mammalian cells are infected with the phage and the cells selected by detection of the marker. The cells that have the most efficient endosomal escape sequence should have the highest expression or most resistance. Multiple rounds of selection may be performed. The peptide genes are recovered and engineered into a phage. The chemical libraries can be peptide libraries, peptidomimetic libraries, other non-peptide synthetic organic libraries, etc.

Exemplary libraries are commercially available from several sources (ArQule, Tripos/PanLabs, ChemDesign, Pharmacopoeia). In some cases, these chemical libraries are generated using combinatorial strategies that encode the identity of each member of the library on a substrate to which the member compound is attached, thus allowing direct and immediate identification of a molecule that is an effective endosome disruptor. Thus, in many combinatorial approaches, the position on a plate of a compound specifies that compound's composition. Also, in one example, a single plate position may have from 1-20 chemicals that can be screened by administration to a well containing the interactions of interest. Thus, if the desired activity is detected, smaller and smaller pools of interacting pairs can be assayed for the activity. By such methods, many candidate molecules can be screened.

Many diversity libraries suitable for use are known in the art and can be used to provide compounds to be tested according to the present invention. Alternatively, libraries can be constructed using standard methods. Chemical (synthetic) libraries, recombinant expression libraries, or polysome-based libraries are exemplary types of libraries that can be used.

The libraries c(an be constrained or semirigid (having some degree of structural rigidity), or linear or nonconstrained. The library can be a cDNA or genomic expression library, random peptide expression library or a chemically synthesized random peptide library, or non-pepticle library. Expression libraries are introduced into the cells in which the assay occurs, where the nucleic acids of the library are expressed to produce their encoded proteins.

In one embodiment, peptide libraries that can be used in the present invention may be libraries that are chemically synthesized in vitro. Examples of such libraries are given in Houghten et al., 1991, Nature 354:84-86, which describes mixtures of free hexapeptides in which the first and second residues in each peptide were individually and specifically defined; Lam et al., 1991, Nature 354:82-84, which describes a “one bead, one peptide” approach in which a solid phase split synthesis scheme produced a library of peptides in which each bead in the collection had immobilized thereon a single, random sequence of amino acid residues; Medynski, 1994, Bio/Technology 12:709-710, which describes split synthesis and T-bag synthesis methods; and Gallop et al., 1994, J. Medicinal Chemistry 37(9):1233-1251. Simply by way of other examples, a combinatorial library may be prepared for use, according to the methods of Ohlmeyer et al., 1993, Proc. Natl. Acad. Sci. USA 90:10922-10926; Erb et al., 1994, Proc. Natl. Acad. Sci. USA 91:11422-11426; Houghten et al., 1992, Biotechniques 13:412; Jayawickreme et al., 1994, Proc. Natl. Acad. Sci. USA 91:1614-1618; or Salmon et al., 1993, Proc. Natl. Acad. Sci. USA 90:11708-11712. PCT Publication No. WO 93/20242 and Brenner and Lerner, 1992, Proc. Natl. Acad. Sci. USA 89:5381-5383 describe “encoded combinatorial chemical libraries,” that contain oligonucleotide identifiers for each chemical polymer library member.

Further, more general, structurally constrained, organic diversity (e.g., nonpeptide) libraries, can also be used. By way of example, a benzodiazepine library (see e.g., Bunin et al., 1994, Proc. Natl. Acad. Sci. USA 91:4708-4712) may be used.

Conformationally constrained libraries that can be used include but are not limited to those containing invariant cysteine residues which, in an oxidizing environment, cross-link by disulfide bonds to form cystines, modified peptides (e.g., incorporating fluorine, metals, isotopic labels, are phosphorylated, etc.), peptides containing one or more non-naturally occurring amino acids, non-peptide structures, and peptides containing a significant fraction of γ-carboxyglutamic acid.

Libraries of non-peptides, e.g., peptide derivatives (for example, that contain one or more non-naturally occurring amino acids) can also be used. One example of these are peptoid libraries (Simon et al., 1992, Proc. Natl. Acad. Sci. USA 89:9367-9371). Peptoids are polymers of non-natural amino acids that have naturally occurring side chains attached not to the alpha carbon but to the backbone amino nitrogen. Since peptoids are not easily degraded by human digestive enzymes, they are advantageously more easily adaptable to drug use. Another example of a library that can be used, in which the amide functionalities in peptides have been permethylated to generate a chemically transformed combinatorial library, is described by Ostresh et al., 1994, Proc. Natl. Acad. Sci. USA 91:11138-11142).

The members of the peptide libraries that can be screened according to the invention are not limited to containing the 20 naturally occurring amino acids. In particular, chemically synthesized libraries and polysome based libraries allow the use of amino acids in addition to the 20 naturally occurring amino acids (by their inclusion in the precursor pool of amino acids used in library production). In specific embodiments, the library members contain one or more non-natural or non-classical amino acids or cyclic peptides. Non-classical amino acids include but are not limited to the D-isomers of the common amino acids, α-amino isobutyric acid, 4-aminobutyric acid, Abu, 2-amino butyric acid; γ-Abu, ε-Ahx, 6-amino hexanoic acid; Aib, 2-amino isobutyric acid; 3-amino propionic acid; ornithine; norleucine; norvaline, hydroxyproline, sarcosine, citrulline, cysteic acid, t-butylglycine, t-butylalanine, phenylglycine, cyclohexylalanine, β-alanine, designer amino acids such as β-methyl amino acids, Cα-methyl amino acids, Nα-methyl amino acids, fluoro-amino acids and amino acid analogs in general. Furthermore, the amino acid can be D (dextrorotary) or L (levorolary).

In addition, or alternatively, membrane disruptive peptides may be expressed and secreted into the endosorne by the attenuated Salmonella vector to assist in the escape of the phage from the endosome.

Another sequence that may be included in the phage is a sequence that facilitates protein trafficking into the nucleus. Such sequences, called nuclear localization signals, are known in the art and are generally rich in positively charged amino acids. Since the carboxyl-terminus of filamentous phage protein VIII is already positively charged, increasing the positive charge increases the likelihood of nuclear transport. Nuclear localization signals can also be fused to other capsid proteins of filamentous phages. The nuclear localization signal fusion can be distinct from the escape moiety fusion. Examples of such nuclear localization -signals include, but are not limited to, the short basic nuclear localization signal of SV 40 T antigen, the bipartite nuclear localization signal of nucleoplasmin, the ribonucleoprotein sequence A1. Moreover, a random peptide library of sequences can be screened fcr novel sequences that promote nuclear localization as described above.

Another sequence that may be included in the phage is a sequence that facilitates internalization of the expressed gene product of interest into a tumor cell. Such sequences, called ferry peptides, are known in the art and have been shown to facilitate the delivery of a polypeptide or peptide of interest to virtually any cell within diffusion limits of its production or introduction (see., e.g., Bayley, 1999, Nature Biotechnology 17:1066-1067; Fernandez et al., 1998, Nature Biotechnology 16:418-420; and Derossi et al., 1998, Trends Cell Biol. 8:84-87). Examples of ferry peptides include the penetratin peptide, which is derived from amino acids 43-58 of helix 3 of the Drosophila melanogaster transcription factor antennapedia (Derossi et al., 1994, J. Biol. Chem. 269:10444-10450; Derossi et al., 1998, Trends Cell Biol. 8:84-87). Yet another exemplary ferry peptide is an 11 amino acid cationic peptide derived from the HIV TAT protein (Schwarze et al., 1999, Science 285:1569-1572). Other examples include, Kaposi fibroblast growth factor (FGF) membrane-translocating sequence (MTS) and herpes simplex virus protein VP22. See, e.g., Bayley, 1999, Nature Biotechnology 17:1066-1067 and Fernandez and Bayley, 1998, Nature Biotechnology 16:418-420 for recent reviews of ferry peptides. Such ferry peptide sequences can be fused to the gene product of interest or to a capsid protein of filamentous phages. Moreover, a random peptide library of sequences can be screened for novel ferry peptide sequences that promote internalization as described above.

### 5.3 Gene Product of Interest

The gene product of interest is selected from the group consisting of proteinaceous and nucleic acid molecules. In various embodiments, the proteinaceous molecule is a cellular toxin (cytotoxic agent), e.g., saporin, cytotoxic necrotic factor-1, cytotoxic necrotic factor-2, a ribosome inactivating protein, or a porin protein, such as gonococcal PI porin protein. In other embodiments, the proteinaceous molecule is an anti-angiogenesis protein or an antibody. In yet other embodiments, the proteinaceous molecule is a cytokine, e.g., IL-2, or a pro-drug converting enzyme, e.g., Herpes Simplex Virus (“HSV”) thymidine kinase or cytosine deaminase. The nucleic acid molecule can be double stranded or single stranded DNA or double stranded or single stranded RNA, as well as triplex nucleic acid molecules. The nucleic acid molecule can function can function as a ribozyme, DNazyme or antisense nucleic acid, etc.

As discussed above, the nucleic acid encoding a gene product of interest is provided in operative linkage with a selected promoter, and optionally in operative linkage with other elements that participate in transcription, translation, localization, stability and the like.

The nucleic acid molecule encoding the gene product of interest is from about 6 base pairs to about 100,000 base pairs in length. Preferably the nucleic acid molecule is from about 20 base pairs to about 50,000 base pairs in length. More preferably the nucleic acid molecule is from about 20 base pairs to about 10,000 base pairs in length. Even more preferably, it is a nucleic acid molecule from about 20 pairs to about 4,000 base pairs in length.

Nucleic acid molecules can encode proteins to replace defective gene products or provide factors to combat certain diseases or syndromes. Many genetic defects are caused by a mutation in a single gene. Introduction of the wild-type gene product will serve to alleviate the deficiency or genetic abnormality. Such gene products include HPRT, adenosine deaminase, LDL receptor, Factor IX, Factor VIII, growth hormone, von Willebrand factor, PTH (parathyroid hormone), M-CSF, TGF-β, PDGF, VEGF, FGF, IGF, BMP (bone morphogenic protein), collagen type VII, fibrillin, Insulin, cystic fibrosis transmembrane conductance regulator, fas ligand, methionase, streptavidin, and the like.

For example, in ischemia, endothelial and smooth muscle cells fail to proliferate. A Salmonella containing phage that expresses FGF, alone or in combination with FGF protein to give short-term relief and induce FGF receptor, can be used to combat effect of ischemia. In such a case, FGF gene open reading frame with a leader sequence to promote secretion is preferable. As well, the expression of FGF is preferably driven by a constitutive promoter.

In addition, certain angiogenic diseases suffer from a paucity of angiogenic factor and thus be deficient in microvessels. Certain aspects of reproduction, such as ovulation, repair of the uterus after menstruation, and placenta development depend on angiogenesis. For reproductive disorders with underlying angiogenic dysfunction, a Salmonella containing phage that expresses FGF, VEGF, or other angiogenic factors, may be beneficial.

Alternatively, in certain diseases such as cancer, angiogenesis is desirably suppressed using anti-angiorganic, factors such as endostatin. Additional exemplary anti-angiogenic factors include, angiostatin, apomigren, anti-angiogenic antithrombin III, the 29 kDa N-terminal and a 40 kDa and/or 29 kDa C-terminal proteolytic fragments of fibronectin, a uPA receptor antagonist, the 16 kDa proteolytic fragment of prolactin, the 7.8 kDa proteolytic fragment of platelet factor-4, the anti-angiogenic 13 amino acid fragment of platelet factor-4, the anti-angiogenic 14 amino acid fragment of collagen I, the anti-angiogenic 19 amino acid peptide fragment of Thrombospondin I, the anti-angiogenic 20 amino acid peptide fragment of SPARC, RGD and NGR containing peptides, the small anti-angiogenic peptides of laminin, fibronectin, procollagen and EGF, and peptide antagonists of integrin αᵥβ₃ and the VEGF receptor. The anti-angiogenic factor can also be a Flt-3 ligand or nucleic acid encoding the same.

Cytokine immumotherapy is a modification of immunogene therapy and involves the administration of tumor cell vaccines that are genetically modified ex vivo or in vivo to express various cytokirie genes. In animal tumor models, cytokine gene transfer resulted in significant antitumor immune response (Fearon, et al., 1990, Cell 60:387-403; Wantanabe, et al., 1989, Proc. Nat. Acad. Sci. USA, 86:9456-9460). Thus, in the present invention, the Salmonella containing phage are used to deliver nucleic acid molecules that encode a cytokine, such as IL-1, IL-2, IL-4, IL-5, IL-15, IL-18, IL-12, IL-10, GM-CSF, INF-γ, INF-α, SLC, endothelial monocyte activating protein-2 (EMAP2), MIP-3α, MIP-3β, or an MHC gene, such as HLA-B7. Addtionally, other exemplary cytokines include members of the TNF family, including but not limited to tumor necrosis factor-α (TNF-α), tumor necrosis factor-β (TNF-β), TNF-α-related apoptosis-inducing ligand (TRAIL), TNF-α-related activation-induced cytokine (TRANCE), TNF-α-related weak inducer of apoptosis (TWEAK), CD40 ligand (CD40L), LT-α, LT-β, OX4OL, CD4OL, FasL, CD27L, CD30L, 4-1BBL, APRIL, LIGHT, TL1, TNFSF16, TNFSF17, and AITR-L, or a functional portion thereof. See, e.g., Kwon et al., 1999, Curr. Opin. Immunol. 11:340-345 for a general review of the TNF family. Delivery of these gene products will modulate the immune system, increasing the potential for host antitumor immunity. Alternatively, nucleic acid molecules encoding costimulatory molecules, such as B7.1 and B7.2, ligands for CD28 and CTLA-4 respectively, can also be delivered to enhance T cell mediated immunity. These gene products can be co-delivered with cytokines, using the same or different promoters and optionally with an internal ribosome binding site. Similarly, α-1,3-galactosyl transferase expression on tumor cells allows complement-mediated cell killing.

As well, acquired or complex multispecific diseases, such as renal failure-induced erythropoietin deficiency, Parkinson's disease (dopamine deficiency), adrenal insufficiency, immune deficiencies, cyclic neutropenia, could be treated using a therapeutic gene product delivered by the vectors of the present invention. In some cases, vascular growth is desirable. As smooth muscle cells underlie the vasculature, delivery of nucleic acid encoding endothelial growth factors, such as FGFs, especially FGF-2, VEGF, tie1, and tie2, through smooth muscle cells is advantageous.

The gene product of interest may also be a bacteriocin (see e.g., Konisky, 1982, Ann. Rev. Microbiol. -26:125-144) which acts as a cytotoxin. In a preferred mode of this embodiment of the invention, the bacteriocin is a colicin, most preferably colicin E3 or V, although colicins A, E1, E2, Ia, Ib, K, L, M (see, Konisky, 1982, Ann. Rev. Microbiol. 36:125-144) can alternatively be used. In another preferred mode of this embodiment, the bacteriocin is a cloacin, most preferably cloacin DF13. The gene product of interest may be another bacteriocin, including but not limited to, pesticin A1122, staphylococcin 1580, butyricin 7423, vibriocin (see e.g, Jayawardene and Farkas-Himsley, 1970, J. Bacteriology vol 102 pp 382-388), pyocin R1 or AP41, and megacin A-216.

For example, Colicin E3 (ColE3) has been shown to have a profoundly cytotoxic effect on mammalian cells (see, Smarda et al, 1978, Folia Microbiol. 23:272-277), including a leukemia cell model system (see, Fiska et al, 1978, Experimentia 35: 406-407). ColE3 cytotoxicity is a function of protein synthesis arrest, mediated by inhibition of 80S ribosomes (Turnowsky et al., 1973, Biochem. Biophys. Res. Comm. 52:327-334). More specifically, ColE3 has ribounclease activity (Saunders, 1978, Nature 274:113-114). In its naturally occurring form, ColE3 is a 60 kDa protein complex consisting of a 50 kDa and a 10 kDa protein in a 1:1 ratio, the larger subunit having the nuclease activity and the smaller subunit having inhibitory function of the 50 kDa subunit. Thus, the 50 kDa protein acts as a cytotoxic protein (or toxin), and the 10 kDa protein acts as an anti-toxin. Accordingly, in one embodiment, when ColE3 is expressed as the protein of interest, the larger ColE3 subunit or an active fragment thereof is expressed alone or at higher levels than the smaller subunit.

Another exemplary bacteriocin is cloacin DF13. Cloacin DF13 functions in an analogous manner to ColE3. The protein complex is of 67 kDa molecular weight. The individual components are 57 kDa and 9 kDa in size. In addition to its ribonuclease activity, DF13 can cause the leakage of cellular potassium. Yet another exemplary bacteriocin is colicin V (see, e.g., Pugsley and Oudega, “Methods for Studing Colicins and their Plasmids” in Plasmids a Practical Approach 1987, ed. by K. G. Hardy; Gilson, L. et al., 1990, EMBO J. 9:3875-3884).

Other bactericcins which may be the gene product of interest according to the present invention include, but are not limited to, colicin E2 (a dual subunit colicin similar to ColE3 in structure but with endonuclease rather than ribonuclease activity); colicins A, E1, Ia, Ib, or K, which form ion-permeable channels, causing a collapse of the proton motive force of the cell and leading to cell death; colicin L which inhibits protein, DNA and RNA synthesis; colicin M which causes cell sepsis by altering the osmotic environment of the cell; pesticin A1122 which functions in a manner similar to colicin B function; staphycoccin 1580, a pore-forming bacteriocin; butyricin 7423 which indirectly inhibits RNA, DNA and protein synthesis through an unknown target; Pyocin P1, or protein resembling a bacteriophage tail protein that kills cells by uncoupling respiration from solute transport; Pyocin AP41 which has a colicin E2-like mode of action; and megacin A-216 which is a phospholipase that causes leakage of intracellular material (for a general review of bacteriocins, see Konisky, 1982, Ann. Rev. Microbiol. 36:125-144).

In a particular embodiment, in which the gene product of interest is a colicin expressed under the control of a SOS-responsive promoter, the attenuated bacterial strain may be treated with x-rays, ultraviolet radiation, an alkylating agent or another DNA damaging agent such that expression of the colicin is increased. Exemplary SOS-responsive promoters include, but are not limited to, recA, sulA, umuC, dinA, ruv, uvrA, uvrB, uvrD, umuD, lexA, cea, caa, recN, etc. See, e.g., Schnarr et al., 1991, Biochimie 73: 423-431 for a general review of SOS-responsive promoters.

The gene product of interest may also be a cytocide, including a pro-drug converting enzyme. A cytocide-encoding agent is a nucleic acid molecule (e.g., DNA or RNA) that, upon internalization by a cell, and subsequent transcription (if DNA) and[/or] translation into a product is cytotoxic or cytostatic to a cell, for example, by inhibiting cell growth through interference with protein synthesis or through disruption of the cell cycle. Such a product may act by cleaving rRNA or ribonucleoprotein, inhibiting an elongation factor, cleaving mRNA, or other mechanism that reduced protein synthesis to a level such that the cell cannot survive.

Examples of suitable gene products include, without limitation, saporin, the ricins, abrin, other ribosome inactiviting proteins (RIPs), Pseudomonas exotoxin, inhibitors of DNA, RNA or protein synthesis, antisense nucleic acids, other metabolic inhibitors (e.g., DNA or RNA cleaving molecules such as DNase and ribonuclease, protease, lipase, phospholipase), prodrug converting enzymes (e.g., thymidine kinase from HSV and bacterial cytosine deaminase), light-activated porphyrin, ricin, ricin A chain, maize RIP, gelonin, E. coli cytotoxic necrotic factor-1, Vibrio fischeri cytotoxic necrotic factor-1, cytotoxic necrotic factor-2, Pasteurella multicida toxin (PMT), cytolethal distending toxin, hemolysin, verotoxin, diphtheria toxin, diphtheria toxin A chain, trichosanthin, tritin, pokeweed antiviral protein (PAP), mirabilis antiviral protein (MAP), Dianthins 32 and 30, abrin, monodrin, bryodin, shiga, a catalytic inhibitor of protein biosynthesis from cucumber seeds (see, e.g., International Publication WO 93/24620), Pseudomonas exotoxin, E. coli heat-labile toxin, E. coli heat-stable toxin, EaggEC stable toxin-1 (EAST), biologically active fragments of cytotoxins and others known to those of skill in the art. See, e.g., O'Brian and Holmes, Protein Toxins of Escherichia coli and Salmonella in Escherichia and Salmonella Cellular and Molecular Biology, Neidhardt et al. (eds.), pp. 2788-2802, ASM Press, Washington, D.C. for a review of E. coli and Salmonella toxins. Yet other exemplary gene products of interest include, but are not limited to, methionase, aspariginase and glycosidase.

Nucleic acid molecules that encode an enzyme that results in cell death or renders a cell susceptible to cell death upon the addition of another product are preferred. Ribosome-inactivating proteins (RIPs), which include ricin, abrin, and saporin, are plant proteins that catalytically inactivate eukaryotic ribosomes. Ribosome-inactivating proteins inactivate ribosomes by interfering with the protein elongation step of protein synthesis. For example, the ribosome-inactivating protein saporin is an enzyme that cleaves rRNA and inhibits protein synthesis. Other enzymes that inhibit protein synthesis are especially well suited for use in the present invention. Any of these proteins, if not derived from mammalian sources, may use mammalian-preferred codons. Preferred codon usage is exemplified in Current Protocols in Molecular Biology, infra, and Zhang et al., 1991, Gene 105:61.

A nucleic acid molecule encoding a pro-drug converting enzyme may alternatively be used within the context of the present invention. Pro-drugs are inactive in the host cell until either a substrate or an activating molecule is provided. As used herein, a “pro-drug converting enzyme” is a compound that metabolizes or otherwise converts an inactive, nontoxic compound to a biologically, pharmaceutically, therapeutically, of toxic active form of the compound or is modified upon administration to yield an active compound through metabolic or other processes. Most typically, a pro-drug converting enzyme activates a compound with little or no cytotoxicity into a toxic compound. Two of the more often used pro-drug converting molecules, both of which are suitable for use in the present invention, are HSV thymidine kinase and E. coli cytosine deaminase.

Briefly, a wide variety of gene products which either directly or indirectly activate a compound with little or no cytotoxicity into a toxic product may be utilized within the context of the present invention. Representative examples of such gene products include HSVTK (herpes simplex virus thymidine kinase) and VZVTK (varicella zoster virus thymidine kinase), which selectively phosphorylate certain purine arabinosides and substituted pyrimidine compounds. Phosphorylation converts these compounds to metabolites that are cytotoxic or cytostatic. For example, exposure of the drug ganciclovir, acyclovir, or any of their analogues (e.g., FIAU, FIAC, DHPG) to cells expressing HSVTK allows conversion of the drug into its corresponding active nucleotide triphosphate form.

Other gene products may be utilized within the context of the present invention include E. coli guanine phosphoribosyl transferase, which converts thioxanthine into toxic thioxanthine monophosphate (Besnard et al., Mol. Cell. Biol. 7: 4139-4141, 1987); alkaline phosphatase, which converts inactive phosphorylated compounds such as mitomycin phosphate and doxorubicin-phosphate to toxic dephosphorylated compounds; fungal (e.g., Fusarium oxysporum) or bacterial cytosine deaminase, which converts 5-fluorocytosine to the toxic compound 5-fluorouracil (Mullen, PNAS, 89:33, 1992); carboxypeptidase G2, which c(leaves glutamic acid from para-N-bis (2-chloroethyl) aminobenzoyl glutamic acid, thereby creating a toxic benzoic acid mustard; and Penicillin-V amidase, which converts phenoxyacetabide derivatives of doxorubicin and melphalan to toxic compounds (see generally, Vrudhula et al., 1993, J. of Med. Chem. 36(7):919-923; Kern et al., 1990, Canc. Immun. Immunother. 31 (4):202-206). Moreover, a wide variety of Herpesviridae thymidine kinases, including both primate and non-primate herpesviruses, are suitable. Such herpesviruses include Herpes Simplex Virus Type 1 (McKnight et al., 1980, Nuc. Acids Res. 8:5940-5946), Herpes Simplex Virus Type 2 (Swain and Galloway, 1983, J. Virol. 46:1045-1050), Varicella Zoster Virus (Davison and Scott, 1986, J. Gen. Virol. 67:1759-1816), marmoset herpesvirus (Otsuka and Kit, 1984, Virology 135:316-330), feline herpesvirus type 1 (Nunberg et al., 1989, J. Virol. 63:3240-3249), pseudorabies virus (Kit and Kit, 1985, U.S. Pat. No. 4,514,497), equine herpesvirus type 1 (Robertson and Whalley, 1988, Nuc. Acids Res. 16:11303-11317), bovine herpesvirus type 1 (Mittal and Field, 1989, J. Virol. 70:2901-2918) turkey herpesvirus (Martin et al., 1989, J. Virol. 63:2847-2852), Marek's disease virus (Scott et al., 1989, J. Gen. Virol. 70:3055-3065), herpesvirus saimiri (Honess et al., 1984, J. Gen. Virol. 70:207-311). Such herpesviruses may be readily obtained from commercial sources such as the American Type culture collection (“ATCC”, Manassas, Va.).

Furthermore, as indicated above, a wide variety of inactive precursors may be converted into active inhibitors. For example, thymidine kinase can phosphorylate nucleosides (e.g. dT) and nucleoside analogues such as ganciclovir (9-{[2-hydroxy-1-(hydroxymethyl)ethoxyl methyl} guanosine), famciclovir, buciclovir, penciclovir, valciclovir, acyclovir (9-[2-hydroxy ethoxy)methyl] guanosine), trifluorothymidine, 1-[2-deoxy, 2-fluor, beta-D-arabino furanosyl]-5-iodouracil, ara-A (adenosine arabinoside, vivarabine), 1-beta-D-arabinofuranoxyl thymine, 5-ethyl-2′-deoxyuridine), AZT (3′ azido-3′ thymidine), ddC (dideoxycytidine), AIU (5-iodo-5′ amino 2′, 5′-dideoxyuridine) and AraC (cytidine arabinoside). Other gene products may render a cell susceptible to toxic agents. Such products include viral proteins, and channel proteins that transport drugs.

Moreover, a cytocide-encoding agent may be constructed as a pro-drug, which when expressed in the proper cell type is processed or modified to an active form.

For example, the saporin gene may be constructed with an N- or C-terminal extension containing a protease-sensitive site. The extension renders the initially translated protein inactive and subsequent cleavage in a cell expressing the appropriate protease restores enzymatic activity.

In a particular embodiment, the gene product of interest comprises a number of viral gene products. For example, the gene product of interest comprises all the viral proteins encoded by an adenovirus or herpesvirus or reovirus genome. In a particular example, the gene product of interest is all the viral proteins encoded by an adenovirus genome except for the E1B viral protein such that this particular adenovirus can only replicate in a mammalian cell lacking p53 activity. Hence in this case the phage genome contains a phage origin of replication and a nucleic acid encoding for all of the adenovirus genome except for E1B. In this particular case wherein the Salmonella containing phage are administered to an organism and delivered to a tumor cell, the produced adenovirus can only replicate in a cell lacking p53 activity, i.e., another tumor cell.

The nucleotide sequences of the genes encoding these gene products are well known (see GenBank). A nucleic acid molecule encoding one of the gene products may be isolated by standard methods, such as amplification (e.g., PCR), probe hybridization of genomic or cDNA libraries, antibody screenings of expression libraries, chemically synthesized or obtained from commercial or other sources.

Additional types of cytocides that may be delivered according to the methods of the present invention are antibody molecules that are preferably expressed within the target cell; hence, these antibody molecules have been given the name “intrabodies.” Conventional methods of antibody preparation and sequencing are useful in the preparation of intrabodies and the nucleic acid sequences encoding same; it is the site of action of intrabodies that confers particular novelty on such molecules. (For a review of various methods and compositions useful in the modulation of protein function in cells via the use of intrabodies, see International Application WO 96/07321).

Intrabodies are antibodies and antibody derivatives (including single-chain antibodies or “SCA”) introduced into cells as transgenes that bind to and incapacitate an intracellular protein in the cell that expresses the antibodies. As used herein, intrabodies encompass monoclonals, single chain antibodies, V regions, and the like, as long as they bind to the target protein. Intrabodies to proteins involved in cell replication, tumorigenesis, and the like (e.g, HER2/neu, VEGF, VEGF receptor, FGF receptor, FGF) are especially useful. The intrabody can also be a bispecific intrabody. Such a bispecific intrabody is engineered to recognize both (1) the desired epitope and (2) one of a variety of “trigger” molecules, e.g., Fc receptors on myeloid cells, and CD3 and CD2 on T cells, that have been identified as being able to cause a cytotoxic T cell to destroy a particular target.

For example, antibodies to HER2/neu (also called erbB-2) may be used to inhibit the function of this protein. HER2/neu has a pivotal role in the progression of certain tumors, human breast, ovarian and non-small lung carcinoma. Thus, inhibiting the function of HER2/neu may result in slowing or halting tumor growth (see, e.g U.S. Pat. No. 5,587,458).

Nucleic acid molecules and oligonucleotides for use as described herein can be synthesized by any method known to those of skill in this art (see, e.g., International Publication WO 93/01286, U.S. Pat. Nos. 5,218,088; 5,175,269; 5,109,124). Identification of oligonucleotides and ribozymes for use as antisense agents and DNA encoding genes for targeted delivery for genetic therapy involve methods well known in the art. For example, the desirable properties, lengths and other characteristics of such oligonucleotides are well known. Antisense oligonucleotides may be designed to resist degradation by endogenous nucleolytic enzymes using linkages such as phosphorothioate, methylphosphonate, sulfone, sulfate, ketyl, phosphorodithioate, phosphoramidate, phosphate esters, and the like (see, e.g., Stein in: Oligodeoxynucleotides, Antisense Inhibitors of Gene Expression, Cohen, Ed, Macmillan Press, London, pp. 97-117, 1989); Jager et al., 1988, Biochemistry 27:7237).

Antisense nucleotides are oligonucleotides that bind in a sequence-specific manner to nucleic acids, such as mRNA or DNA. When bound to mRNA that has complementary sequences, antisense prevents translation of the mRNA (see, e.g., U.S. Pat. Nos. 5,168,053; 5,190,931; 5,135,917; 5,087,617). Triplex molecules refer to single DNA strands that bind duplex DNA forming a colinear triplex molecule, thereby preventing transcription (see, e.g., U.S. Pat. No. 5,176,996).

Particularly useful antisense nucleotides and triplex molecules are molecules that are complementary to bind to the sense strand of DNA or mRNA that encodes a protein involved in cell proliferation, such as an oncogene or growth factor, (e.g., bFGF, int-2, hst-1/K-FGF, FGF-5, hst-2/FGF-6, FGF-8). Other useful antisense oligonucleotides include those that are specific for IL,-8 (see, e.g., U.S. Pat. No. 5,241,049), c-src, c-fos H-ras (lung cancer), K-ras (breast cancer), urokinase (melanoma), BCL2 (T-cell lymphoma), IGF-1 (glioblastoma), IGF-1 (glioblastoma), IGF-1 receptor (glioblastoma), TGF-β1, and CRIPTO EGF receptor (colon cancer). These particular antisense plasmids reduce tumorigenicity in athymic and syngeneic mice.

A ribozyme is an RNA molecule that specifically cleaves RNA substrates, such as mRNA, resulting in inhibition or interference with cell growth or expression. There are at least five known classes of ribozymes involved in the cleavage and/or ligation of RNA chains. Ribozymes can be targeted to any RNA transcript and can catalytically cleave that transcript (see, e.g., U.S. Pat. Nos. 5,272,262; 5,144,019; 5,168,053, 5,180,818, 5,116,742 and 5,093,246).

In addition, inhibitors of inducible nitric oxide synthase (NOS) and endothelial nitic oxide synthase are cytocides that are useful for delivery to cells. Nitric oxide (NO) is implicated to be involved in the regulation of vascular growth and tone in arterosclerosis. NO is formed from L-arginine by nitric oxide synthase (NOS) and modulates immune, inflammatory and cardiovascular responses.

In one embodiment, the nucleic acid molecule encodes for an antigen. The antigen can be a tumor-associated antigen or the antigen can be associated with an infectious agent. An example of a tumor-associated antigen is a molecule specifically expressed by a tumor cell and is not expressed in the non-cancerous counterpart cell or is expressed in the tumor cell at a higher level than in the non-cancerous counterpart cell. Illustrative examples of tumor associated antigens are described in Kuby, Immunology, W. H. Freeman and Company, New York, N.Y., pp. 515-520 and Robbins and Kawakami, 1996, Curr. Opin. Immunol. 8:628-363, which are incorporated by reference herein, and include melanocyte lineage proteins such as gp100, MART-1MelanA, TRP-1 (gp75), tyrosinase; tumor-specific, widely shared antigens such as MAGE-1, MAGE-3, BAGE, GAGE-1, -2, N-acetylglucosaminyltransferase-V, p15; tumor-specific, mutated antigens such as β-catenin, MUM-1, CDK4; and non-melanoma antigens such as HER-2/neu (breast and ovarian carcinoma), human papilloma virus-E6, E7 (cervical carcinoma), MUC-1 (breast, ovarian and pancreatic carcinoma). Other examples of tumor associated antigens are known to those of skill in the art.

Useful antigens associated with an infectious agent include, but are not limited to, antigens from pathogenic strains of bacteria (Streptococcus pyogenes, Streptococcus pneumoniae, Neisseria gonorrhoea, Neisseria meningitidis, Corynebacterium diphtheriae, Clostridium botulinum, Clostridium perfringens, Clostridium tetani, Haemophilus influenzae, Klebsiella pneumoniae, Klebsiella ozaenae, Klebsiella rhinoscleromotis, Staphylococcus aureus, Vibrio cholerae, Escherichia coli, Pseudomonas aeruginosa, Campylobacter (Vibrio) jejuni, Aeromonas hydrophila, Bacillus cereus, Edwardsiella tarda, Yersinia enterocolitica, Yersinia pestis, Yersinia pseudotuberculosis, Shigella dysenteriae, Shigella flexneri, Shigella sonnei, Salmonella typhimurium, Treponema pallidum, Treponema pertenue, Treponema carateneum, Borrelia vincentii, Borrelia burgdorferi, Leptospira icterohemorrhagiae, Mycobacterium tuberculosis, Toxoplasma gondii, Pneumocystis carinii, Francisella tularensis, Brucella abortus, Brucella suis, Brucella melitensis, Mycoplasma spp., Rickettsia prowazeki, Rickettsia tsutsugumushi, Chlamydia spp., Helicobacter pylori); pathogenic fungi (Coccidioides immitis, Aspergillus fumigatus, Candida albicans, Blastomyces dermatitidis, Cryptococcus neoformans, Histoplasma capsulatum); protozoa (Entomoeba histolytica, Trichomonas tenas, Trichomonas hominis, Trichomonas vaginalis, Trypanosoma gambiense, Trypanosoma rhodesiense, Trypanosoma cruzi, Leishmania donovani, Leishmania tropica, Leishmania braziliensis, Pneumocystis pneumonia, Plasmodium vivax, Plasmodium falciparum, Plasmodium malaria); or Helminiths (Enterobius vermicularis, Trichuris trichiura, Ascaris lumbricoides, Trichinella spiralis, Strongyloides stercoralis, Schistosoma japonicum, Schistosoma mansoni, Schistosoma haematobium, and hookworms).

Other relevant infectious agent antigens are pathogenic viruses (as examples and not by limitation: Poxviridae, Herpesviridae, Herpes Simplex virus 1, Herpes Simplex virus 2, Adenoviridae, Papovaviridae, Enteroviridae, Picornaviridae, Parvoviridae, Reoviridae, Retroviridae, influenza viruses, parainfluenza viruses, mumps, measles, respiratory syncytial virus, rubella, Arboviridae, Rhabdoviridae, Arenaviridae, Hepatitis A virus, Hepatitis B virus, Hepatitis C virus, Hepatitis E virus, Non-A/Non-B Hepatitis virus, Rhinoviridae, Coronaviridae, Rotoviridae, and Human Immunodeficiency Virus). Other examples of antigens associated with infectious agents are known to those of skill in the art.

### 5.4 Methods and Compositions for Delivery

According to the present invention, the attenuated, optionally tumor-targeting, Salmonella vectors containing a bacteriophage encoding a gene product of interest are advantageously used in methods for delivery of an agent, or in methods for inducing an immune response, or in methods to produce a tumor growth inhibitory response or a reduction of tumor volume in an animal including a human patient having a solid tumor cancer. In one embodiment of the present invention, a method for delivery of an agent comprises administering, to a subject, a pharmaceutical composition comprising an effective amount of an attenuated Salmonella containing a bacteriophage wherein the bacteriophage genome has been modified to encode for a gene product of interest under the control of an appropriate eukaryotic promoter or wherein the genome of the bacteriophage has been modified to encode the gene of interest as a fusion protein with a bacteriophage capsid protein, e.g., phage protein III or VIII. In one embodiment of the invention, a method for inducing an immune response in subject to an antigen comprises administering, to a subject, a pharmaceutical composition comprising an effective amount of an attenuated Salmonella containing a bacteriophage wherein the bacteriophage genome has been modified to encode for an antigen under the control of an appropriate eukaryotic promoter or wherein the genome of the bacteriophage has been modified to expresses the antigen as a fusion with a bacteriophage capsid protein. In yet another embodiment of the present invention, a method of treating solid tumors comprises administering, to a subject in need of such treatment, a pharmaceutical composition comprising an effective amount of an attenuated, tumor-targeting Salmonella containing a bacteriophage wherein the bacteriophage genome has been modified to encode for a gene product of interest under the control of an appropriate eukaryotic promoter or wherein the genome of the bacteriophage has been modified to encode for a gene of interest as a fusion protein with a bacteriophage capsid protein, e.g., phage protein III or VIII. Solid tumors include, but are not limited to, sarcomas, carcinomas or other solid tumor cancers, such as germ line tumors and tumors of the central nervous system, including, but not limited to, breast cancer, prostate cancer, cervical cancer, uterine cancer, lung cancer, ovarian cancer, testicular cancer, thyroid cancer, astrocytoma, glioma, pancreatic cancer, stomach cancer, liver cancer, colon cancer, and melanoma. The subject is preferably an animal, including but not limited to animals such as cows, pigs, chickens, etc., and is preferably a mammal, and most preferably human. Effective treatment of a solid tumor, includes but is not limited to, inhibiting tumor growth, reducing tumor volume.

In an alternative embodiment of the present invention, an attenuated, optionally tumor-targeting Salmonella vector expressing the F′ pilus is administered to the subject separately from the filamentous bacteriophage. The bacteriophage can be administered, prior to, concurrently or after administration of the Salmonella vector.

The amount of the pharmaceutical composition of the invention which will be effective in the treatment or prevention of a particular disorder or condition will depend on the nature of the disorder or condition, and can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. However, suitable dosage ranges are generally from about 1.0 c.f.u./kg to about 1×10¹⁰ c.f.u./kg; optionally from about 1.0 c.f.u./kg to about 1×10⁸ c.f.u./kg; optionally from about 1×10² c.f.u./kg to about 1×10⁸ c.f.u./kg; optionally from about 1×10⁴ c.f.u./kg to about 1×10⁸ c.f.u./kg. Effective doses may be extrapolated from dose-response curves derived from in vitro or animal model test systems.

Various delivery systems are known and can be used to administer a pharmaceutical composition of the present invention. Methods of introduction include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved by, for example, and not by way of limitation, local infusion during surgery, by injection, by means of a catheter, or by means of an implant, said implant being of a porous, non-porous, or gelatinous material, including membranes, such as sialastic membranes, or fibers. In one embodiment, administration can be by direct injection at the site (or former site) of a malignant tumor or neoplastic or pre-neoplastic tissue.

In another embodiment, the Salmonella vector and/or bacteriophage can be delivered in a controlled release system. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)).

Other controlled release systems are discussed in the review by Langer (Science 249:1527-1533 (1990)).

The present invention is also directed to a pharmaceutical composition comprising a pharmaceutically acceptable carrier and an attenuated, and optionally, tumor-targeting, Gram-negative bacterial vector, such as Salmonella or Shigella spp., containing a bacteriophage, wherein the genome of the bacteriophage has been modified to encode for a gene product of interest under the control of an appropriate eukaryotic promoter or wherein the genome of the bacteriophage has been modified to encode for a gene of interest as a fusion protein with a bacteriophage capsid protein, e.g., phage protein III or VIII. Such compositions comprise a therapeutically effective amount of a Salmonella vector, and a pharmaceutically acceptable carrier. The present invention is also directed to a pharmaceutical composition comprising a pharmaceutically acceptable carrier and an attenuated, and optionally, tumor-targeting, Salmonella vector expressing the F′ pilus and a pharmaceutical composition comprising a filamentous bacteriophage, wherein the genome of the bacteriophage has been modified to encode for a gene product of interest under the control of an appropriate eukaryotic promoter or wherein the genome of the bacteriophage has been modified to encode for a gene of interest as a fusion protein with a bacteriophage capsid protein, e.g., phage protein III or VIII. Such compositions comprise a therapeutically effective amount of a Salmonella vector or filamentous bacteriophage, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids., such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose., magnesium carbonate, etc. Examples of suitable pharmaceutical carriers are described in “Remington's Pharmaceutical Sciences” by E. W. Martin. Such compositions will contain a therapeutically effective amount of the Therapeutic, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The invention also provides a pharmaceutical pack or kit comprising one or more containers filled with one or more of the ingredients of the pharmaceutical compositions of the invention. For example, the kit can comprise two vials, one containing a pharmaceutical composition comprising an attenuated, optionally tumor-targeting, Salmonella vector expressing the F′ pilus and the other vial containing a pharmaceutical composition comprising a filamentous bacteriophage, wherein the genome of the bacteriophage has been modified to encode for a gene product of interest under the control of an appropriate eukaryotic promoter or wherein the genome of the bacteriophage has been modified to encode for a gene of interest as a fusion protein with a bacteriophage capsid protein, e.g., phage protein III or VIII. Optionally associated with such container(s) can be instructions for use of the kit and/or a notice in the form prescribed by a governmental agency regulating the manufacture, use or sale of pharmaceuticals or biological products, which notice reflects approval by the agency of manufacture, use or sale for human administration.

The following series of examples are presented by way of illustration and not by way of limitation on the scope of the invention.

### 6. EXAMPLE

### Salmonella Expressing Phage

The following series of experiments demonstrate that an attenuated, tumor-targeting Salmonella containing a bacteriophage genome, which genome has a nucleic acid which encodes for a gene product of interest, can deliver the gene product of interest to mammalian cells, leading to expression of the gene product of interest in the mammalian cells.

### 6.1. Expression of Phagemid DNA in Mammalian Cells

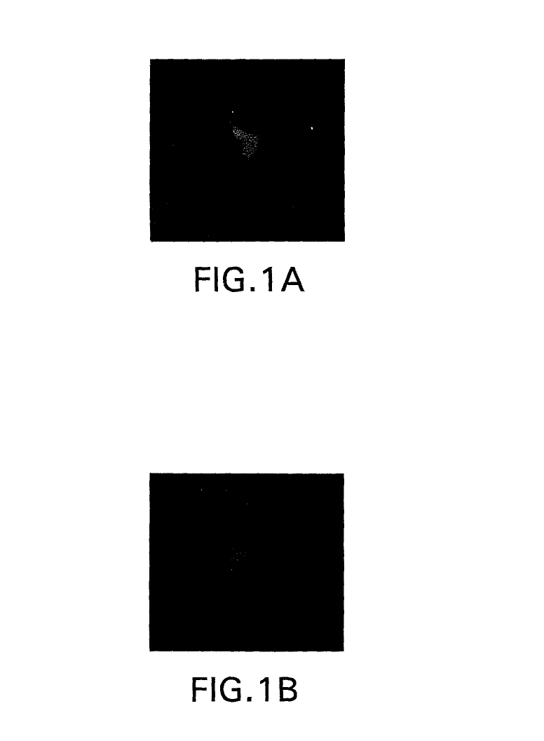

Single-stranded phagemid DNA, designated pBSKIIGFP, which encodes for green fluorescent protein (“GFP”) under the control of an eukaryotic-specific promoter (CMV promoter) was isolated from Salmonella and transiently transfected into mouse COS 7 cells using SUPERFECT™ obtained from Stratagene (LaJolla, Calif.). GFP was used as a model for a gene product of interest. The results are shown in FIGS. 1A and 1B.

FIGS. 1A and 1B show that the encoded GFP was successfully expressed in COS 7 cells containing pBSKIIGFP, indicating that single stranded DNA phagemid molecules isolated from Salmonella can be used to express a phage genome-encoded gene product in mammalian cells.

### 6.2. Salmonella Delivery of Phage

Once it was shown that a gene could be expressed from a single-stranded DNA phagemid in an eukaryotic cell, it was next asked whether the phagemid could be transfected into the cells using Salmonella.

Salmonella strain 72 (see International Publication WO 96/40238, which is incorporated by reference, for a description of strain 72) was engineered to express the F′ pilus as follows such that the strain is able to be infected by phage. Salmonella strain YS501 (recD⁻, chloramphenicol resistant) was mated with E. coli strain NH4104, which carries the F′ plasmid containing the lactose operon, and Salmonella colonies were selected for chloramphenicol resistance, lac⁺ on minimal media containing lactose and chloramphenicol. This strain, designated YS501-F′ (which is also met⁻) was mated with Salmonella strain 72 (which is also pur⁻) and Salmonella colonies were selected on minimal media containing lactose and purine but lacking methionine. This strain was designated 72-F′.

Salmonella strains VNP20009 and YS1456 were also engineered to express the F′ pilus according to the same method described in the preceding paragraph.

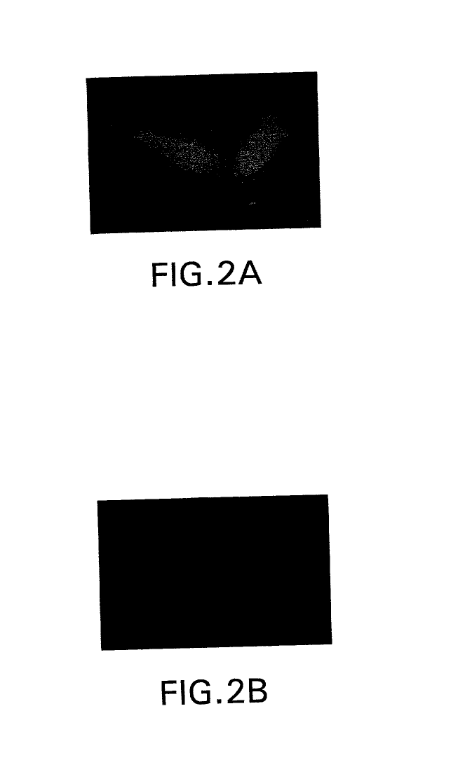

Salmonella strain 72-F′ was then infected with M13KO7 helper phage and the resultant Salmonella strain, 72-F′-M13KO7, which was selected by kanamycin resistance, was infected with pBSKIIGFP. Salmonella strain 72-F′-Mi3KO7 infected with pBSKIIGFP was used to infect mammalian M2 cells as follows. Approximately 2×10⁷ c.f.u. Salmonella were incubated with M2 cells for one hour at 37° C. in cell culture medium. The M2 cells were washed twice with fresh medium containing 100 μg/ml gentamycin and incubated for another hour at 37° C. in medium containing 100 μg/ml gentamycin. The cell culture medium was replaced with fresh medium containing 100 μg/ml gentamycin and the cells were incubated further overnight at 37° C. After overnight incubation, the M2 cells were analyzed for the presence of DNA in the cytoplasm and GFP expression. The cells were also stained either with DAPI or propidum iodide (PI) to stain the nuclei and any bacteria in the cytoplasm. The results are shown in FIGS. 2A and 2B.

FIG. 2A shows that M2 cells infected with the phage containing Salmonella strain expressed GFP. FIG. 2B shows no GFP expression in M2 cells not infected with the phage containing Salmonella. These results demonstrate that mammalian cells can be successfully transfected with Salmonella containing phage and express a phage genome-encoded gene product.

### 6.3 Phage Infected Tumor-Targeting Salmonella

The following, experiment shows that a tumor-targeting strain of Salmonella retains the ability to target tumors when infected with phage, and that viable phage particles can be recovered from the soluble supernatant fraction of the tumor demonstrating that the phage is replicated and released by the Salmonella at the tumor site.

Salmonella strain 72-F′-M13KO7 was injected into two C57BL6 mice containing B16F10 melanoma tumors at a titer of 4×10⁵ c.f.u. per mouse. On day 4 after injection, the mice were sacrificed and the livers and tumors were harvested and homogenized on ice. Various dilutions of the homogenate were plated directly onto LB medium to determine the tumor targeting ability of the strain. An aliquot of the homogenate was centrifuged for 15 minutes at 12,000 rpm, the supernatant was removed and centrifuged for an additional 15 minutes at 12,000 rpm. The resulting supernatant at various dilutions was plated onto LB medium to determine bacterial carryover. An aliquot of the supernatant was also used to determine phage recovery by infecting F′ pilus expressing E. coli cells (JM 109) and plating the infected bacteria on selective media to score for phage infection. The results are presented in Table 1.

TABLE 1

| | c.f.u./ml | c.f.u./gram | tumor:liver |
|---|---|---|---|
| A. Bacteria in homogenate: | | | |
| Tumor mouse 1 | 1.7 × 10⁹ | 4.4 × 10⁹ | 400:1 |
| Liver mouse 1 | 1.9 × 10⁶ | 1.1 × 10⁷ | |
| Tumor mouse 2 (DEAD) | 2.9 × 10⁸ | 7.5 × 10⁸ | 1:1 |
| Liver mouse 2 | 1.2 × 10⁸ | 7.2 × 10⁸ | |
| B. Bacteria in supernatant | | | |
| Tumor mouse 1 | 2.2 × 10⁵ | | |
| Liver mouse 1 | 0 | | |
| Tumor mouse 2 (DEAD) | 4.1 × 10⁴ | | |
| Liver mouse 2 | 0 | | |
| | p.f.u./ml | p.f.u./gram | corrected* |
| C. Phage in supernatant | | | |
| Tumor mouse 1 | 3.5 × 10⁸ | 8.7 × 10⁸ | 4.6 × 10¹¹ |
| Liver mouse 1 | 9.0 × 10⁵ | 5.0 × 10⁶ | 2.6 × 10⁹ |
| Tumor mouse 2 (DEAD) | 7.0 × 10⁶ | 1.7 × 10⁷ | 8.9 × 10⁹ |
| Liver mouse 2 | 4.6 × 10⁵ | 2.7 × 10⁶ | 1.4 × 10⁹ |
| Control (1.0 × 10¹¹) | 1.9 × 10⁸* | | |

*Approximately 526 fold less p.f.u. was recovered than expected from the control infection experiment, due to the low infectivity of JM109 cells. This value was used as a correction factor to generate the number listed in the right column.

The results clearly show that phage can be delivered to the tumor without disrupting the tumor-targeting ability of the Salmonella vector.

### 6.4 Phage Infected Tumor-Targeting, Attenuated Salmonella

The following experiment clearly shows that an attenuated, tumor-targeting Salmonella strain, msbB⁻ 8.7 (see International Publication WO 99/13053, which is incorporated by reference, for a description of strain msbB⁻) can deliver phage to tumors.

Salmonella strain msbB⁻ 8.7 was engineered to express the F′ pilus as follows such that the strain is able to be infected by phage. Salmonella strain YS501-F′ (which is also met⁻), described in Section 6.2, supra, was mated with Salmonella strain msbB⁻ 8.7 (which is also pur⁻) and selected on minimal media containing lactose and purine but lacking methionine. This strain was designated msbB⁻ 8.7-F′. This strain was then infected with M13KO7 and the resultant Salmonella strain, msbB⁻ 8.7-F′-M13KO7, was selected by kanamycin resistance. The msbB⁻ 8.7-F′-M13KO7 strain was then injected into five C57BL6 mice containing B16F10 melanoma tumors at a titer of2×10⁶ c.f.u. per mouse. The number of bacteria and phage in the liver and tumor homogenates was determined as described in Section 6.3, supra. The results are shown in Table 2.

TABLE 2
A. Bacteria in homogenate

| Tumors: | | Livers: | |
|---|---|---|---|
| Animal No. | c.f.u./gram | Animal No. | c.f.u./gram |
| 1 | 4.0 × 10⁹ | 1 | 1.3 × 10⁷ |
| 2 | 3.6 × 10⁹ | 2 | 4.2 × 10⁷ |
| 3 | 4.3 × 10⁹ | 3 | 0.52 × 10⁷ |
| 4 | 4.5 × 10⁹ | 4 | 1.0 × 10⁷ |
| 5 | 4.8 × 10⁹ | 5 | 1.6 × 10⁸ |
| Tumors | Livers | Tumor:liver | |
| 4.2 ± 0.4 × 10⁹ | 4.6 ± 5 × 10⁷ | 91:1 (with No.5) | |
| | 1.7 ± 1.4 × 10⁷ | 248:1 (without No.5) | |

B. Phage in supernatant*

| Tumors: | | Livers | |
|---|---|---|---|
| Animal No. | p.f.u./gram | Animal No. | p.f.u./gram |
| 1 | 17 × 10⁹ | 1 | 2.5 × 10⁵ |
| 2 | 2.7 × 10⁹ | 2 | 23 × 10⁵ |
| 3 | 0 | 3 | 0.58 × 10⁵ |
| 4 | 5.4 × 10⁹ | 4 | 0 |
| 5 | 5.9 × 10⁹ | 5 | 9.4 × 10⁵ |
| Tumors | Livers | Tumor:liver | |
| 6.3 ± 5.7 × 10⁹ | 7.2 ± 8.6 × 10⁵ | 8750:1 | |

*Correction factor for phage recovery: 4.5

The results clearly show that an attenuated, tumor-targeting Salmonella vector can deliver phage to tumors.

### 7. EXAMPLE

### Production of IL-2 Phage

The following experiment demonstrates that Salmonella-produced phage with a tripartite interleukin-2 (IL-2)-OmpA-8L-pIII fusion protein produced phage particles which possess IL-2 activity.

A fusion of IL-2 to a phage pIII protein was produced in phagemid pSKAN8 (MoBiTec, Marco Island, Fla.). A modified ompA signal peptide (OmpA-8L) containing amino acid substitutions within and flanking the ten amino acid hydrophobic core of the signal sequence was demonstrated to be capable of facilitating the secretion of IL-2 from Salmonella when fused to the amino terminus of IL-2. The DNA encoding this sequence, along with the wild type ompA sequence, is depicted below.

Wild Type ompA

| | |
|---|---|
| Wild type ompA | (SEQ ID NO:3) |

5′-ATGAAAAAGACAGCTATCGCGATTGCAGTGGCACTGGCTGGTTTCGC
TACCGTAGCGCAGGCC-3′

(SEQ ID NO:4)

MKKTAIAIAVALAGFATVAQA

Mutant OmpA-8L

| | |
|---|---|
| Mutant OmpA-8L | (SEQ ID NO:5) |

5′-ATGAAAAAGACGGCTCTGGCGCTTCTGCTCTTGCTGTTAGCGCTGAC
TAGTGTAGCGCAGGCC-3′

(SEQ ID NO:6)

MKKTALALLLLLLALTSVAQA

A Sal I-EcoRV fragment encompassing the ompA-hPSTI gene fusion from pSKAN8 (Bio101, Vista, Calif.) was removed from the phagemid and replaced with the ompA-8L-IL-2 gene fusion generated by PCR from the following oligonucleotides using an ompA8L-IL-2 fusion plasmid as the template:

(SEQ ID NO:7)

5′-gcGTCGACcaaggaggtctagataacgagggcaaaaaATGAAAAAGA
CGGCTCTGGCGCTTCTG-3′ and

(SEQ ID NO:8)

5′-gcgaattcGATATCTTCAGTTAACGTGCTAATGATCGATTGG-3′

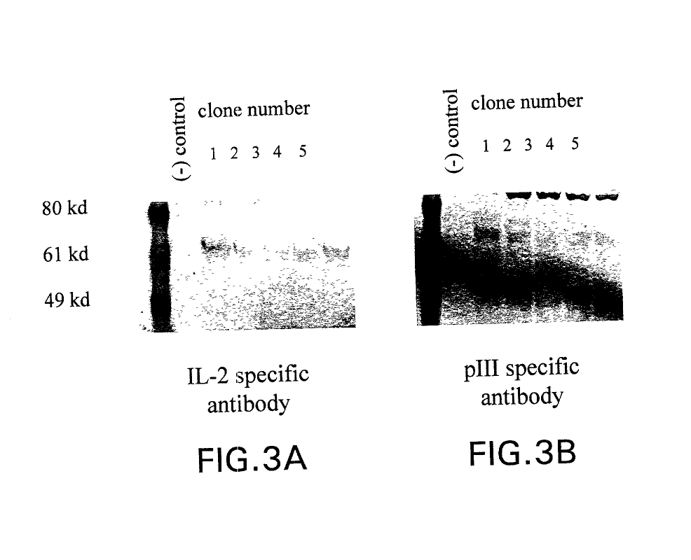

The PCR-generated fragment was subcloned into pSKAN8 such that the final amino acid encoding codon of IL-2 was in frame with the pIII gene in pSKAN8, which resulted in a gene fusion between IL-2 and pIII. Upon expression of this fusion in E. coli, a protein of the expected molecular weight of the protein fusion (62 kd) was observed in a Western analysis utilizing antibodies to either IL-2 or pIII (FIGS. 3A-3B).

To demonstrate that the fusion protein could be packaged into phage particles produced from Salmonella and that these phage particles possessed IL-2 activity, two strains of Salmonella carrying an M13KO7 helper phage were transformed with the pSKAN8-IL-2::pIII phagemid, with the subsequent production and secretion of phage particles. Purified phage particles were examined for IL-2 activity (compared to helper phage alone) using an IL-2-dependent mouse cytotoxic T cell line, CTLL-2, in a proliferation assay as described in Gearing and Bird, In: Lymphokines and Interferons, A Practical Approach, Clemens et al. (eds.), IRL Press, p. 296.

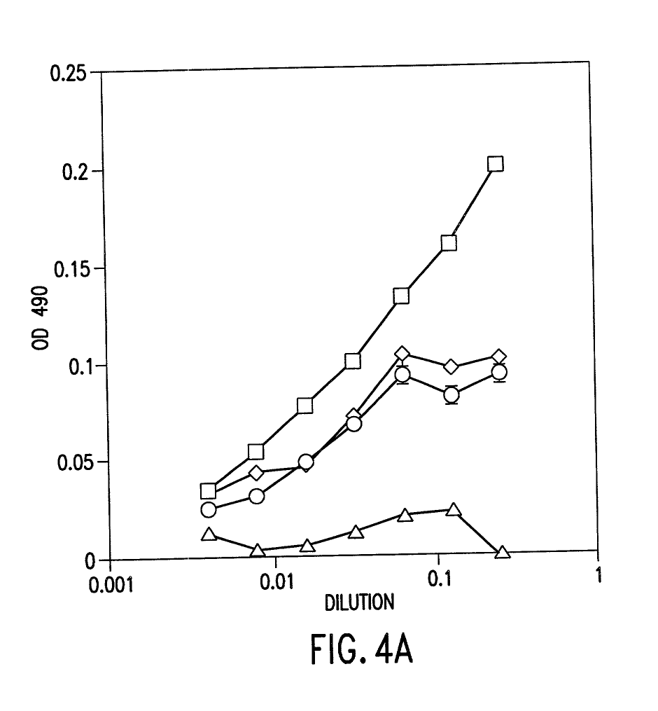

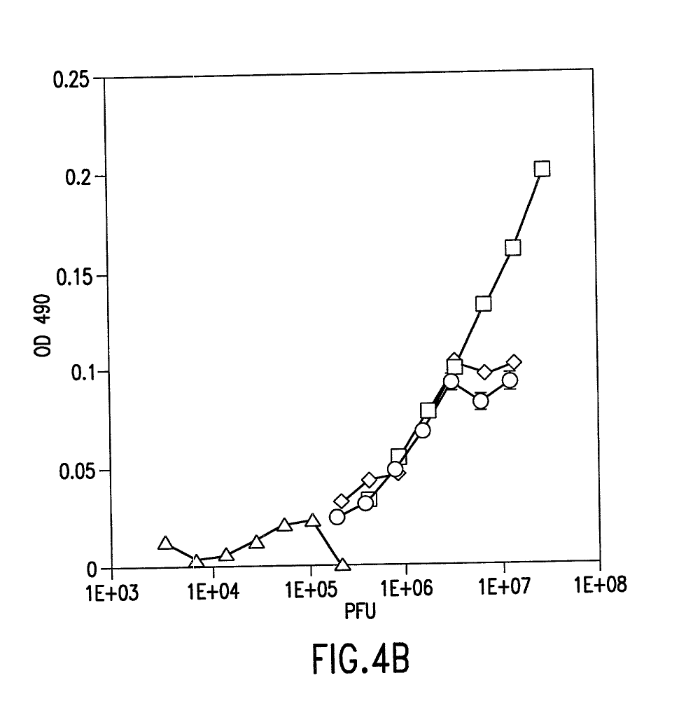

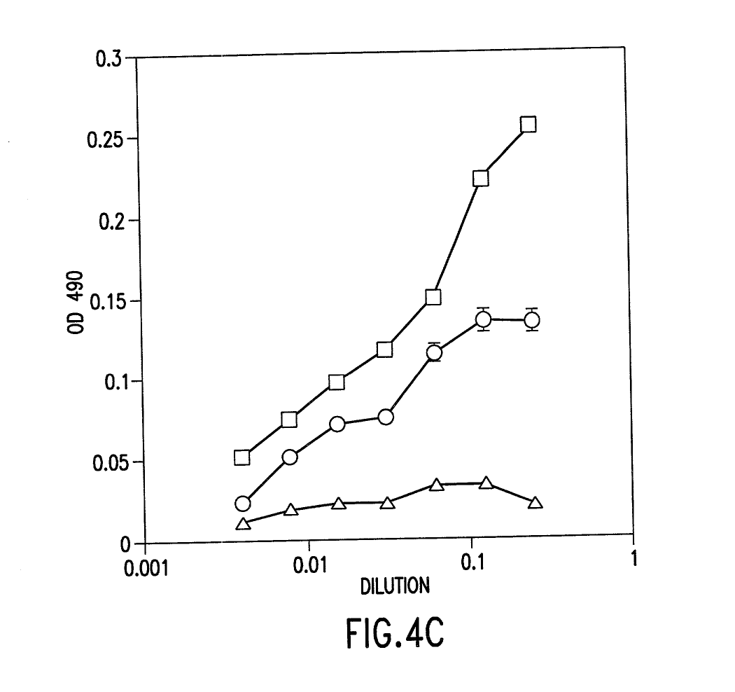

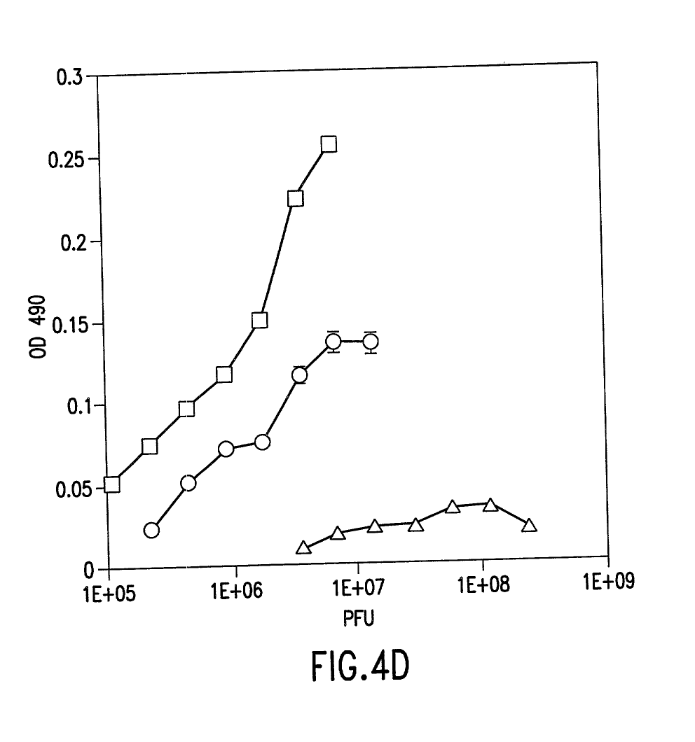

As indicated in FIGS. 4A-4B, phage particles produced from Salmonella strain 41.2.9 carrying the Phagemid in three separate experiments possessed significant IL-2 activity in a concentration dependent manner (open circle, open square, open diamond), whereas phage particles produced from the Salmonella strain carrying only helper phagemid did not possess IL-2 activity (open triangle). FIGS. 4C-4D demonstrate the same results with a different Salmonella strain, 8.7.

### 8. EXAMPLE

### Cloning of Listeriolysin O in Phage

The following experiment describes the cloning of listeriolysin O 91-99 for DNA delivery by a bacteriophage produced by Salmonella. Listeriolysin O (LLO) is a secreted protein from L. monocytogenes and is processed by a host cell through the classical MHC class I processing pathway which, results in a CTL response. It has been shown that an epitope comprising of amino acids 91-99 of LLO elicits a strong CTL response. LLO 91-99 is an illustrative example of a protein which can be delivered to tumor cells by Salmonella-producing phage according to the present invention. Delivery and expression of LLO 91-99 into mammalian cells and subsequent processing and presentation of the LLO 91-99 peptide can be tested in vitro by standard CTL assays or by FACS.

The LLO 91-9:9 peptide (GYKDGNEYI) (SEQ ID NO: 9) was codon-optimized and synthesized using complimentary oligonucleotides. At the 5′ end sequence encoding for additional 6 LLO amino acids, an Spe I site, a start codon and the Kozac consensus was added. At the 3′ end sequence for 6 LLO amino acids, a stop codon and a Not I site were added: LLO5F:

| | |
|---|---|
| LLO5F: | (SEQ ID NO:10) |

5′-GCCACCATGACTAGTAATGTGCCGCCGCGTAAAGGTTACAAAGATGG
TAATGAATATATCGTTGTGGAGAAAAAGAAATAGGCGGCCGCAAAAGGAA
AA-3′

| | |
|---|---|
| LLO6R: | (SEQ ID NO:11) |

5-′TTTTCCTTTTGCGGCCGCCTATTTCTTTTTCTCCACAACGATATATT
CATTACCATCTTTGTAACCTTTACGCGGCGGCACATTACTAGTCATGGTG
GC-3′

The two oligos were annealed to give the double stranded fragment:

5′-GCCACC ATG ACTAGT AATGTGCCGCCGCGTAAAGGTTACAAAGA
3′-CGGTGG TAC TGATCA TTACACGGCGGCGCATTTCCAATGTTTCT
TGGTAATGAATATATCGTTGTGGAGAAAAAGAAATAGG CGGCCG CAAA
ACCATTACTTATATAGCAACACCTCTTTTTCTTTATCC GCCGGC GTTT
AGGAAAA-3′
TCCTTTT-5′

(Restriction sites are italicized and the Kozac consensus is bolded.)

After a restriction digest, the fragment was cloned into the Sma I/Not I restricted phagemid pEGFP-N1 (Clontech, Palo Alto, Calif.). The construct has been confirmed by DNA sequencing.

The Phagemid is transformed into Salmonella along with helper phage M13KO7 for the generation of phage particles that package the LLO DNA.

### 9. EXAMPLE

### Cloning of HIV TAT Ferry Peptide

The following example describes the cloning of the 11 amino acid ferry peptide derived from the HIV TAT protein into phage. The 11 amino acid HIV TAT peptide and the TAT peptide with a hexahistidine tag (TAT6H) (Schwarze et al., 1999, Science 285:1569-1572) were cloned by PCR using overlapping primers as a self-template. The TAT sequence was obtained from Genbank Accession number AAA81040.1 (Collman, et al., 1992, J. Virol. 66:7517-7521) and the amino acid sequence reverse translated using codons frequently used by Salmonella (see, e.g., Current Protocols in Molecular Biology, infra, and Zhang et al., 1991. Gene 105: 61). Primers used to clone TAT by PCR were Phage TAT F1B 5′-GATCACrATCTTATGGCCGCAAAAAACGCCG-3′ (SEQ ID NO: 12), which contains a Bg/II site and Phage TAT R1B 5′-TATGGCCGCAAAAAAICGCCGTCAGCGCCGTCGCGAGCTCGATC-3′ (SEQ ID NO: 13) which contains a SacI site. Primers used for TAT6H were Phage6H-TAT F1B 5 ′-GATCAGATCTCATCACCATCACCACCATTATGGCCGCAAAAAACGCCGT-3′ (SEQ ID NO: 14), which contains a Bg/II and Phage TAT R1B (SEQ ID NO: 13). The PCR products were cut with Bg/II/SacI and ligated to the Gene III region of pHage3.2 (phagemid derived from M13, Maxim Biotech, Inc., So. San Francisco, Calif.) prepared by cutting with Bg/II/SacI. The sequence was verified at the Yale University Keck sequencing center.

Phage3.2 6HTAT was cut with PvuII. The 1664 bp+/−20 bp DNA piece was isolated and ligated to the StuI site of pEGFP-N1 phagemid (Clonetech, Palo Alto, Calif.). The clones obtained were screened for orientation by HindIII digests and also cut with NruI to detect the NruI site in TAT and TAT6H sequence. A TAT6H clone with the correct sequence was transformed into VNP20009 containing the F′ pilus. One these clones was then infected with the helper phage R408 (Stratagene, La Jolla, Calif.). The subsequent clones of VNP20009 containing the F′ pilus, the pHage 6HTAT fusions in the Gene III region subcloned into the pEGFPN1 (kanamycin resistance), and the R408 helper phage were screened to determine the production of 6HTAT-modified phagemids by taking culture supernatants filtered through a 0.2 um filter and incubating with VNP20009 containing the F′ pilus (kanamycin sensitive) in order to allow phagemid infection of the bacteria, thus carrying in the antibiotic resistance marker and then plating for kanamycin resistant colonies which contain the phagemid. One clone (TAT6H3 clone 1.1) was found to produce 10 million phagemid particles per ml. These results demonstrate that a tumor specific bacterium can produce phagemids with a modification of the gene III protein containing the hexahistidine-TAT sequence.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

Various publications are cited herein, the disclosures of which are incorporated by reference in their entireties.

What is claimed is:

1. A pharmaceutical composition comprising a pharmaceutically acceptable carrier and an attenuated, tumor-targeting Gram-negative bacterium containing a bacteriophage, wherein the genome of the bacteriophage has been modified to encode for a gene product of interest under the control of an eukaryotic promoter or wherein the genome of the bacteriophage has been modified to encode the gene of interest as a fusion protein with a bacteriophage capsid protein.

2. The composition according to claim 1 in which the bacterium is a Salmonella.

3. The composition according to claim 1 in which the Gram-negative bacterium is Shigella.

4. The composition according to claim 1 in which the gene product of interest is a proteinaceous molecule.

5. The composition according to claim 1 in which the gene product of interest is an antigen.

6. The composition according to claim 4 in which the molecule is selected from the group consisting a cytokine, a cytotoxin, a pro-drug converting enzyme and an anti-angiogenic agent.

7. The composition according to claim 6 in which the cytotoxin is a bacteriocin.

8. A kit comprising an attenuated, tumor-targeting Gram-negative bacterium containing a bacteriophage, wherein the genome of the bacteriophage has been modified to encode for a gene product of interest under the control of an eukaryotic promoter or wherein the genome of the bacteriophage has been modified to encode the gene of interest as a fusion protein with a bacteriophage capsid protein, together with instructions for administering the attenuated, tumor-targeting Gram-negative bacterium containing a bacteriophage to a subject to deliver the gene product of interest.

9. A kit comprising an attenuated, tumor-targeting Gram-negative bacterium expressing the F′ pilus and a filamentous bacteriophage, wherein the genome of the bacteriophage has been modified to encode for a gene product of interest under the control of an eukaryotic promoter or wherein the genome of the bacteriophage has been modified to encode the gene of interest as a fusion protein with a bacteriophage capsid protein, together with instructions for administering the attenuated, tumor-targeting Gram-negative bacterium expressing the F′ pilus and a filamentous bacteriophage to a subject to deliver the gene product of interest.

10. The kit according to claim 8 or 9 in which the Gram-negative bacterium is Salmonella or Shigella.

11. A method for delivering an agent comprising administering, to a subject, a pharmaceutical composition comprising an attenuated Gram-negative bacterium containing a bacteriophage, wherein the bacteriophage genome has been modified to encode for a gene product of interest under the control of an eukaryotic promoter or wherein the genome of the bacteriophage has been modified to encode for a gene of interest as a fusion protein with a bacteriophage capsid protein.

12. The method according to claim 11, in which the gene of interest is an antigen or a pro-drug converting enzyme.

13. The method according to claim 11, in which the gene of interest is fused with a bacteriophage capsid protein.

14. A method for delivering an agent comprising administering, to a subject, a pharmaceutical composition comprising an attenuated Gram-negative bacterium expressing the F′ pilus and a filamentous bacteriophage, wherein the bacteriophage genome has been modified to encode for a gene product of interest under the control of an eukaryotic promoter or wherein the genome of the bacteriophage has been modified to encode for a gene of interest as a fusion protein with a bacteriophage capsid protein.

15. A method of inhibiting tumor growth or reducing tumor volume comprising administering, to a subject in need of such inhibition or reduction, a pharmaceutical composition comprising an attenuated, tumor-targeting Gram-negative bacterium containing a bacteriophage, wherein the bacteriophage genome has been modified to encode for a gene product of interest under the control of an eukaryotic promoter or wherein the genome of the bacteriophage has been modified to encode the gene of interest as a fusion protein with a bacteriophage capsid protein.

16. The method according to claim 15 in which the Gram-negative bacterium is Salmonella or Shigella.

17. A method of inhibiting tumor growth or reducing tumor volume comprising administering, to a subject in need of such inhibition or reduction, a pharmaceutical composition comprising an attenuated, tumor-targeting Gram-negative bacterium expressing the F′ pilus and a bacteriophage, wherein the bacteriophage genome has been modified to encode for a gene product of interest under the control of an eukaryotic promoter or wherein the genome of the bacteriophage has been modified to encode the gene of interest as a fusion protein with a bacteriophage capsid protein.

18. The method according to claim 17 in which the Gram-negative bacterium is Salmonella or Shigella.

19. The method according to claim 15 or 17 in which the solid tumors is selected from the group consisting of breast cancer, prostate cancer, cervical cancer, uterine cancer, lung cancer, ovarian cancer, testicular cancer, thyroid cancer, astrocytoma, glioma, mesothelioma, renal cancer, bladder cancer, pancreatic cancer, stomach cancer, liver cancer, colon cancer, and melanoma.

20. The method according to claim 11 or 15 in which the bacteriophage is a phagemid.

21. The method according to claim 11 or 15 in which the gene product of interest is a proteinaceous molecule.

22. The method according to claim 21 in which the molecule is selected from the group consisting a cytokine, a cytotoxin, a pro-drug converting enzyme and an anti-angiogenic agent.

23. The method according to claim 21 in which the gene product of interest is a proteinaceous molecule fused to a ferry peptide sequence.

24. An attenuated, tumor targeting Salmonella expressing the F′ pilus.
